(19) United States
(12) Patent Application Publication
(10) Pub. No.: US 2024/0335584 A1
(43) Pub. Date: Oct. 10, 2024

(54) NONABSORBABLE SETTABLE MULTI-PUTTY BONE CEMENTS, HEMOSTATIC COMPOSITIONS AND METHODS OF USE

(71) Applicant: Abyrx, Inc., Stamford, CT (US)

(72) Inventors: Aniq DARR, Riverdale, NY (US); Bryant HEIMBACH, Stamford, CT (US); John PACIFICO, Greenwich, CT (US); Richard L. KRONENTHAL, Fair Lawn, NJ (US)

(21) Appl. No.: 18/294,895

(22) PCT Filed: Jul. 29, 2022
(86) PCT No.: PCT/US2022/038801

§ 371 Date: Feb. 2, 2024

Related U.S. Application Data
(60) Provisional application No. 63/229,209, filed on Aug. 4, 2021.

(51) Int. Cl. A61L 24/00; C04B 26/16; C04B 111/00

(52) U.S. Cl. CPC A61L 24/0084; A61L 24/0089; C04B 26/16; A61L 2430/02; C04B 2111/00836

(57) ABSTRACT

Provided herein are settable, nonabsorbable bone hemostatic and adhesive compositions for use in surgical procedures comprising a variety of disclosed particles. Also provided are related compositions, including surgical kits and packages, as well as methods of making and using the compositions.

### RELATED APPLICATIONS

This application claims priority to U.S. Patent Application No. 63/229,209 filed Aug. 4, 2021, the contents of which are herein incorporated by reference in their entirety.

### FIELD OF THE INVENTION

The present disclosure provides multiple-putty, polyurethane, polyureaurethane, polyetherurethane and polyetherureaurethane-based compositions for use in hemostasis, bone repair or reconstruction, that remain nonabsorbable after implantation. The composition of the present disclosure comprises two or more putties that are mixed just prior to surgical use, wherein the mixing results in a reaction of the at least two components forming a nonabsorbable and settable hemostatic putty composition. Also provided herein are methods for using the mixed putty composite in surgical applications to repair gaps and/or fractures or to provide tissue adhesion.

### BACKGROUND

Bone cements are used during surgery to help anchor implants to natural bone during bone repair and reconstruction. Currently, the most common bone cement used clinically is polymethylmethacrylate (PMMA) and its monomer, methylmethacrylate (MMA), that reacts with a polymerization catalyst. While PMMA is ubiquitously used as a bone cement, there are several disadvantages. Most notably, the reaction of MMA with the catalyst results in a reaction exotherm, with temperatures exceeding 80° C., that can cause damage to surrounding tissue. Polyurethane elastomers known in the art have been increasing in usage as biomaterials because of their appropriate mechanical properties and chemical versatility. Their usage extends to tissue engineering scaffolds (WO 2004009227), in situ tissue repair (U.S. Pat. No. 6,306,177), and bone hemostats (U.S. Pat. No. 9,314,547). The preparation of implantable polyurethane-based compositions require a simple, conventional mix that involves combining pre-weighed amounts of a polyisocyanate, polyol or polyamine, chain extender, and, optionally, filler materials that typically consist of bioceramics, polymers, and/or cellulosic materials. Additionally, an antibacterial agent, such as Doxycycline, can be added to the mixture to reduce the occurrence of post-operative infection. The primary components of the polyurethane mixture are typically liquid at room temperature; however, the mixture can include both liquid and solid components depending on the filler materials employed. Settable polymeric cements that are used clinically rely on the in situ hardening of liquid components, requiring mixing of the liquid components in the surgical setting. Materials provided this way are difficult to apply, often becoming slippery when exposed to body fluids and frequently sticking to gloves, surgical equipment, and fixation devices such as wires, plates, and screws. Such complications during implantation can result in polymer misapplication and may cause damage to medical devices, such as drains and catheters, during their removal.

Despite the recent development in the field of polyurethane-based medical devices, namely with the advent of Montage settable absorbable hemostatic bone putty, there remains a need for a polyurethane-based bone cement or bone hemostat that is not hydrolytically degraded in vivo. The present disclosure provides polyurethane-based compositions that provide the handling and material properties needed for bone cements and hemostatic devices while eliminating the detrimental effects of the bone cements known in the art.

### SUMMARY OF THE INVENTION

The present disclosure provides a sterile, settable, nonabsorbable composition comprising a set of at least two reactive putties (i.e., a putty A and at least putty B), which can be hand-mixed and where the putties comprise amounts of reagents which react and cure into a final, hardened form selected from the group consisting of polyurethane, polyureaurethane, polyetherurethane and polyetherureaurethane compositions, over a period of time at room or body temperature, each putty being physically separated from the other putty of the composition; wherein putty A comprises a polyisocyanate component, a polyol or a polyamine component, a non-hydrolysable crosslinker, a particulate material, and additive material(s); and wherein putty B comprises a polyisocyanate component, a polyol and/or a polyamine component, a nonabsorbable cross-linker, a particulate material, and additive material(s).

The present disclosure provides a settable, nonabsorbable composition comprising a set of at least two reactive putties, A and at least B, which, upon mixing together, react and cure into a final hardened form selected from the group consisting of polyurethane, polyureaurethane, polyetherurethane and polyetherureaurethane compositions, over a period of time at room or body temperature, each putty being physically separated from the other putty of the composition; wherein putty A comprises 10-70% of a polyisocyanate component, 0-5% of a polyol or a polyamine component, 30-85% of one or more particulate material(s), and 0-8% of one or more additive material(s), based upon the total weight of putty A; and wherein putty B comprises 0-5% of an polyisocyanate component, 5-80% of a polyol or a polyamine component, 30-95% of one or more particulate material(s), and 0-5% of one or more additive material(s), based upon the weight of putty B, wherein the polyol or polyamine component of putty A or putty B or both comprises one or more nonabsorbable, non-hydrolysable cross-linker(s).

The present disclosure also provides a method of stabilizing a bone fracture or reapproximating a sternotomy, the method comprising the steps of: a) intraoperatively mixing or kneading together a set of at least two reactive putties, A and at least B of the composition disclosed herein, to form a moldable, settable, nonabsorbable polyurethane, polyureaurethane, polyetherurethane or polyetherureaurethane composition at room or body temperature, b) applying the mixed or kneaded composition to the surfaces of the bone fracture or the cut surfaces of the sternotomy and manually reducing the bone fragments while allowing the composition to set; and c) allowing the composition to harden into its fully cured, solid form.

A method of stabilizing a surgical hardware for stabilizing, repairing, or reapproximating a bone fracture, the method comprising the steps of: a) intraoperatively mixing or kneading together a set of at least two reactive putties, A and at least B, of the composition disclosed herein, to form a moldable, settable, nonabsorbable polyurethane, polyureaurethane, polyetherurethane or polyetherureaurethane composition at room or body temperature, b) combining the composition with the surgical hardware, and/or applying the mixed or kneaded composition to the surfaces of the bone fracture or the cut surfaces of the sternotomy, and manually reducing the bone fragments while allowing the composition to set; and c) allowing the composition to harden into its fully cured solid form.

The present disclosure also provides a plurality of biocompatible, settable putties which, upon mixing, react to form a cured final composition at room or body temperature over a period time, the final composition being nonabsorbable under physiological conditions, wherein the plurality of putties, comprises the at least two putties of the composition disclosed herein.

The present disclosure also provides a settable, nonabsorbable putty composition formed by mixing the at least two putties of the present disclosure.

Any of the aspects and embodiments described herein can be combined with any other aspect or embodiment as disclosed here in the Summary of the Invention, in the Drawings, and/or in the Detailed Description of the Invention, including the below specific, non-limiting examples/embodiments of the present invention.

Unless otherwise defined, all technical and scientific terms used herein have the same meaning as commonly understood by one of ordinary skill in the art to which this application belongs. In the specification, the singular forms also include the plural unless the context clearly dictates otherwise.

Although methods and materials similar to, or equivalent to, those described herein can be used in the practice and testing of the application, suitable methods and materials are described below. All publications, patent applications, patents, and other references mentioned herein are incorporated by reference.

The references cited herein are not admitted to be prior art to the claimed application. In the case of conflict, the present specification, including definitions, will control. In addition, the materials, methods, and examples are illustrative only and are not intended to be limiting.

Other features and advantages of the application will become apparent from the following detailed description in conjunction with the examples.

### DETAILED DESCRIPTION OF THE INVENTION

### Definitions

In this disclosure, “comprises,” “comprising,” “containing,” “having,” and the like can have the meaning ascribed to them in U.S. Patent law and can mean “includes,” “including,” and the like; the terms “consisting essentially of” or “consists essentially” likewise have the meaning ascribed in U.S. Patent law and these terms are open-ended, allowing for the presence of more than that which is recited so long as basic or novel characteristics of that which is recited are not changed by the presence of more than that which is recited, but excludes prior art embodiments.

Unless specifically stated or obvious from context, as used herein, the terms “a,” “an,” and “the” are understood to be singular or plural.

Unless specifically stated or obvious from context, as used herein, the term “or” is understood to be inclusive.

As used herein, the term “about,” unless indicated otherwise, refers to the recited value, e.g., amount, dose, temperature, time, percentage, etc., ±10%, ±9%, ±8%, ±7%, ±6%, ±5%, ±4%, ±3%, ±2%, or ±1%.

As used herein, the terms “patient” or “subject” are used interchangeably herein to refer to any mammal, including humans, domestic and farm animals, and zoo, sports, and pet animals, such as dogs, horses, cats, and agricultural use animals including cattle, sheep, pigs, and goats. One preferred mammal is a human, including adults, children, and the elderly. A subject may also be a pet animal, including dogs, cats, and horses. Preferred agricultural animals would be pigs, cattle, and goats.

The phrases “therapeutically effective amount” and “effective amount” and the like, as used herein, indicate an amount necessary to administer to a patient, or to a cell, tissue, or organ of a patient, to achieve a therapeutic effect, such as an ameliorating or, alternatively, a curative effect. The effective amount is sufficient to elicit the biological or medical response of a cell, tissue, system, animal, or human that is being sought by a researcher, veterinarian, medical doctor, or clinician. Determination of the appropriate effective amount or therapeutically effective amount is within the routine level of skill in the art.

The term “putty” and the likes refer to soft, moldable, cohesive compositions, as used herein refers to most often formed viscous suspensions or viscoelastic composites (i.e., dispersions of particles in a viscous fluid). The present invention may also be formed from monolithic compositions of waxes and soft polymers: the putties of the invention are distinguished from the transitional “taffy” phases which occur during the setting process of polyurethane, polyureaurethane, polyetherurethane or polyetherureaurethane and other settable compositions.

The present disclosure provides settable, nonabsorbable polyurethane, polyureaurethane, polyetherurethane or polyetherureaurethane compositions. The settable, nonabsorbable polyurethane, polyureaurethane, polyetherurethane or polyetherureaurethane compositions of the present disclosure can be made by combining a polyisocyanate or a polyisocyanate-based prepolymer component with a polyol and/or polyamine component, to form a polyurethane, polyureaurethane, polyetherurethane or polyetherureaurethane-based composition. In some embodiments, the polyisocyanate component and the polyol and/or polyamine component can be combined with one or more chain extenders as described herein.

The settable, nonabsorbable polyurethane, polyureaurethane, polyetherurethane or polyetherureaurethane compositions of the present disclosure are not absorbable in vivo, but are biocompatible and suitable for use in vivo, especially for use in bone repair and replacement surgery. The settable, nonabsorbable polyurethane, polyureaurethane, polyetherurethane or polyetherureaurethane compositions of the present disclosure are suitable for use as a bone cement, bone substitute, and/or a bone hemostatic agent. The term “biocompatible” as used throughout herein refers to materials that do not induce adverse side effects when administered or implanted in vivo. The term biocompatible may be used interchangeably with “nontoxic” herein. The terms “nonabsorbable”, “nondegradable”, and “permanent”, as used throughout herein, are used to describe the persistence of the device once implanted in vivo, and are used interchangeably to refer to the present invention's resistance to degradation.

The present disclosure provides a sterile, settable, nonabsorbable composition comprising a set of at least two reactive putties, A and at least B (i.e., a putty A and at least a putty B), which can be hand-mixed and wherein the putties comprise amounts of reagents which react and cure into a final, hardened form selected from the group consisting of polyurethane, polyureaurethane, polyetherurethane and polyetherureaurethane, over a period of time at room or body temperature, each putty being physically separated from the other putty of the composition; wherein putty A comprises of a polyisocyanate component, a polyol or a polyamine component, a non-hydrolysable crosslinker, a particulate material, and additive material(s); and wherein putty B comprises of a polyisocyanate component, a polyol and/or a polyamine component, a nonabsorbable cross-linker, a particulate material, and additive material(s). In some embodiments, the settable, nonabsorbable polyurethane, polyureaurethane, polyetherurethane or polyetherureaurethane compositions of the present disclosure, are formed by the reaction of one or more polyaromatic di- or polyisocyanates with one or more diols or polyols and/or diamines or polyamines. In some embodiments, the settable, nonabsorbable polyurethane, polyureaurethane, polyetherurethane or polyetherureaurethane compositions of the present disclosure, the compositions of the invention may also include the addition of an optional chain extender or crosslinker. In some embodiments, the settable, nonabsorbable polyurethane, polyureaurethane, polyetherurethane or polyetherureaurethane compositions of the present disclosure, are formed in the absence of a crosslinker.

The present disclosure provides a settable, nonabsorbable composition comprising a set of at least two reactive putties, A and at least B, which, upon mixing together, react and cure into a final hardened form selected from the group consisting of polyurethane, polyureaurethane, polyetherurethane or polyetherureaurethane, over a period of time at room or body temperature, each putty being physically separated from the other putty of the composition; wherein putty A comprises 10-70% of a polyisocyanate component, 0-5% of a polyol or a polyamine component, 30-85% of one or more particulate material(s), and 0-8% of one or more additive material(s), based upon total weight of putty A; and wherein putty B comprises 0-5% of a polyisocyanate component, 5-80% of a polyol or a polyamine component, 30-95% of one or more particulate material(s), and 0-5% of one or more additive material(s), based upon the weight of putty B, wherein the polyol or polyamine component of putty A or putty B or both comprise one or more nonabsorbable, non-hydrolysable crosslinker(s).

In some embodiments of the composition of this disclosure, putty A comprises 25-60% of a polyisocyanate component, and 40-75% of one or more particulate materials(s); based upon the weight of putty A; and putty B comprises 30-70% of a polyol and/or a polyamine component, and 30-70% of one or more particulate material(s), based upon the weight of putty B.

In some embodiments of the composition of this disclosure, putty A comprises 25-60% (e.g., 25%-30%, 30%-35%, 35%-40%, 40%-45%, 45%-50%, 50%-55% or 55%-60%) of a polyisocyanate component, based upon total weight of putty A. In some embodiments of the composition of this disclosure, putty A comprises 40-75% (e.g., 40%-45%, 45%-50%, 50%-55%, 55%-60%, 60%-65%, 65%-70% or 70%-75%) of one or more particulate material(s), based upon total weight of putty A.

In some embodiments of the composition of this disclosure, putty B comprises 30-70% (e.g., 30%-35%, 35%-40%, 40%-45%, 45%-50%, 50%-55%, 55%-60%, 60%-65% or 65%-70%) of a polyol and/or a polyamine component, based upon total weight of putty B. In some embodiments of the composition of this disclosure, putty B comprises 30-70% (e.g., 30%-35%, 35%-40%, 40%-45%, 45%-50%, 50%-55%, 55%-60%, 60%-65% or 65%-70%) of one or more particulate material(s), based upon total weight of putty B.

In some embodiments of the composition of this disclosure, putty A comprises 15-40% of a polyisocyanate component, 2-3% of a polyol and/or a polyamine component, and 60-85% of one or more particulate material(s), based upon the weight of putty A; and putty B comprises 2-5% of a polyisocyanate compound, 15-25% of a polyol and/or a polyamine component, and 70-85% of one or more particulate material(s), based upon the weight of putty B.

In some embodiments of the composition of this disclosure, putty A comprises 15-40% (e.g., 15%-20%, 20%-25%, 25%-30%, 30%-35%, or 35%-40%) of a polyisocyanate component, based upon the total weight of putty A. In some embodiments of the composition of this disclosure, putty B comprises 2%-5% (e.g., 2%-2.5%, 2.5%-3%, 3%-3.5%, 3.5%-4%, 4%-4.5% or 4.5%-5%) of a polyisocyanate component, based upon the total weight of putty B.

In some embodiments of the composition of this disclosure, putty A comprises 2-3% (e.g., 2%-2.2%, 2.2%-2.4%, 2.4%-2.6%, 2.6%-2.8% or 2.8%-3%) of a polyol and/or a polyamine component, based upon the total weight of putty A. In some embodiments of the composition of this disclosure, putty B comprises 15-25% (e.g., 15-16%, 16%-17%, 17%-18%, 18%-20%, 20%-21%, 21%-22%, 22%-23%, 23%-24% or 24%-25%) of a polyol and/or a polyamine component, based upon the total weight of putty B.

In some embodiments of the composition of this disclosure, putty A comprises 60-85% (e.g., 60%-65%, 65%-70%, 70%-75%, 75%-80% or 80%-85%) of one or more particulate materials), based upon the total weight of putty A. In some embodiments of the composition of this disclosure, putty B comprises 70-85% (e.g., 70%-72%, 72%-74%, 74%-76%, 76%-80%, 80%-82% or 82%-85%) of one or more particulate material(s), based upon the total weight of putty B.

In some embodiments of the composition of this disclosure, putty A comprises 25-45% of a polyisocyanate component, 3-5% of a polyol and/or a polyamine component, and 50-65% of one or more particulate material(s), based upon the weight of putty A; and putty B comprises 2-5% of a polyisocyanate compound, 5-35% of a polyol and/or a polyamine component, and 60-92% of one or more particulate material(s), based upon the weight of putty B.

In some embodiments of the composition of this disclosure, putty A comprises 25-45% (e.g., 25%-30%, 30%-35%, 35%-40%, 40%-45% or 45%-50%) of a polyisocyanate component, based upon the total weight of putty A. In some embodiments of the composition of this disclosure, putty B comprises 2-5% (e.g., 2%-2.5%, 2.5%-3%, 3%-3.5%, 3.5%-4%, 4%-4.5% or 4.5%-5%) of a polyisocyanate component, based upon the total weight of putty B.

In some embodiments of the composition of this disclosure, putty A comprises 3-5% (e.g., 3%-3.5%, 3.5%-4%, 4%-4.5% or 4.5%-5%) of a polyol and/or a polyamine component, based upon the total weight of putty A. In some embodiments of the composition of this disclosure, putty B comprises 5-35% (e.g., 5%-10%, 10%-15%, 15%-20%, 20%-25%, 25%-30% or 30%-35%) of a polyol and/or a polyamine component, based upon the total weight of putty B.

In some embodiments of the composition of this disclosure, putty A comprises 50-65% (e.g., 50-52%, 52%-54%, 54%-56%, 56%-58%, 58%-60%, 60%-62% or 62%-65%) of one or more particulate material(s), based upon the total weight of putty A. In some embodiments of the composition of this disclosure, putty B comprises 60-92% (e.g., 60%-64%, 64%-68%, 68%-72%, 72%-76%, 76%-80%, 80%-84%, 84%-88% or 88%-92%) of one or more particulate material(s), based upon the total weight of putty B.

In some embodiments of the composition of this disclosure, putty A comprises 0-8% (e.g., 0-1%, 1%-2%, 2%-3%, 3%-4%, 4%-5%, 5%-6%, 6%-7% or 7%-8%) of one or more additive material(s), based upon the total weight of putty A.

In some embodiments of the composition of this disclosure, putty B comprises 0-5% (e.g., 0-1%, 1%-2%, 2%-3%, 3%-4% or 4%-5%) of one or more additive material(s), based upon the total weight of putty B.

In some embodiments of the composition of this disclosure, the polyisocyanate component of the putty A, the putty B, or both is a nonabsorbable polyisocyanate. In some embodiments of the composition of this disclosure, the polyisocyanate component of the putty A, the putty B, or both is a nondegradable polyaromatic isocyanate, preferably a diisocyanate or a polyisocyanate.

In some embodiments of the composition of this disclosure, the mixture cures into a final hardened composition at either room temperature or body temperature, over a period of time, and without the need to apply additional external heat in excess of the ambient heat of the room, which is about 24-26° C. (e.g., 24° C.-25° C. or 25° C.-26° C.) or the heat of the human body (about 37° C.). In one embodiment, the period of time for the mixture to finish curing into a hardened final form is from about 6 to 12 hours (e.g., 6-7, 7-8, 8-9, 9-10, 10-11 or 11-12 hours) or from about 6 to 24 hours (e.g., 6-7, 7-8, 8-9, 9-10, 10-11, 11-12, 12-13, 13-14, 14-15, 15-16, 16-17, 17-18, 18-19, 19-20, 20-21, 21-22, 22-23, or 23-24 hours). In one embodiment, the fully cured composition is drillable or machinable. In one embodiment, the homogenous composition is thermoplastic and can be softened to return it to a hand-moldable state by applying heat sufficient to warm the composition to a temperature at least higher than 40° C., preferably between 40° C. and 100° C. (i.e., 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100° C.). In accordance with this embodiment, where the composition sets or cures prematurely during use, it may be heated until it becomes moldable again for a period of time until it cools.

In some embodiments of the composition of this disclosure, the polyisocyanate component of the putty A, the putty B, or both comprises one or more one or more polyisocyanate(s). In some embodiments of the composition of this disclosure, the one or more polyisocyanate(s) is a non-hydrolysable polyisocyanate. In some embodiments of the composition of this disclosure, the one or more polyisocyanate(s) of the putty A, the putty B, or both is a diisocyanate, triisocyanate, or tetraisocyanate or a combination thereof. In some embodiments of the composition of this disclosure, the one or more polyisocyanate(s) of the putty A, the putty B, or both is 1,1,1-tris-(4-isocyanatophenoxymethyl)-propane (also referred to as trimethylolpropane-4-nitrophenyl, or TMPI).

In some embodiments of the composition of this disclosure, the polyamine component of the putty A, the putty B or both comprises one or more polyamine. In some embodiments of the composition of this disclosure, the one or more polyamine(s) of the putty A, the putty B, or both is ethylene diamine, propane diamine, butane diamine, cyclopentane diamine, cyclohexane diamine, or hexamethylene diamine, or a combination thereof.

In some embodiments of the composition of this disclosure, the polyol component of the putty A, the putty B or both comprises one or more polyol(s). In some embodiments of the composition of this disclosure, the one or more polyols of the putty A, the putty B, or both is a trimethylolpropane ethoxylate, triethanolamine, tetrakis (2-hydroxyethyl)ethylenediamine, or tetrakis (2-hydroxypropyl)ethylenediamine, or a combination thereof.

In some embodiments of the composition of this disclosure, the one or more nonabsorbable, non-hydrolysable cross-linker(s) or chain extender(s) of the putty A, the putty B, or both is an aliphatic polyol. In some embodiments of the composition of this disclosure, the one or more nonabsorbable, non-hydrolysable crosslinker(s) or chain extender(s) of the putty A, the putty B, or both is a trimethylolpropane ethoxylate, triethanolamine, glycerol, pentaerythritol, a trifunctional castor oil-based polyol, trimethylolpropane polyol, tetrakis(2-hydroxyethyl)ethylenediamine, or tetrakis(2-hydroxypropyl)ethylenediamine, or a combination thereof. In some embodiments of the composition of this disclosure, the trimethylolpropane ethoxylate is a trimethylolpropane ethoxylate (TMPE) of molecular weight 450, a TMPE of molecular weight 170, or a TMPE of molecular weight 1014.

In some embodiments of the composition of this disclosure, the polyisocyanate component and the polyol/polyamine component of the putty A, the putty B, or both of the settable, nonabsorbable polyurethane, polyureaurethane, polyetherurethane or polyetherureaurethane compositions of the present disclosure are crosslinked. In some embodiments of the composition of this disclosure, the polyisocyanate component and the polyol/polyamine component the putty A, the putty B, or both of the settable, nonabsorbable polyurethane, polyureaurethane, polyetherurethane or polyetherureaurethane compositions of the present disclosure are not crosslinked. In some embodiments, the putty A, the putty B, or both of the settable, nonabsorbable polyurethane, polyureaurethane, polyetherurethane or polyetherureaurethane compositions of the invention comprise an ether-linked tri- or tetra-isocyanate and a trimethylolpropane polyol. In some embodiments of the composition of this disclosure, the putty A, the putty B, or both of the settable, nonabsorbable polyurethane, polyureaurethane, polyetherurethane or polyetherureaurethane compositions further comprises triethanolamine as a chain extender. In some embodiments of the composition of this disclosure, the putty A, the putty B, or both of the settable, nonabsorbable polyurethane, polyureaurethane, polyetherurethane or polyetherureaurethane compositions further comprises a tetraol, such as tetrakis(2-hydroxyethyl)ethylenediamine or tetrakis(2-hydroxypropyl)ethylenediamine, as a chain extender and/or a reaction catalyst. In some embodiments of the composition of this disclosure, the putty A, the putty B, or both of the settable, nonabsorbable polyurethane, polyureaurethane, polyetherurethane or polyetherureaurethane compositions further comprises one or more of water, a carboxylic acid (e.g., lauric acid), and/or a divalent or polyvalent metal salt.

In some embodiments of the composition of this disclosure, the particulate material(s) of the putty A, the putty B, or both is a calcium phosphate, siliconized calcium phosphate, substituted calcium phosphates (substituted with magnesium, strontium, or silicate), calcium pyrophosphate, hydroxyapatite, polymethyl methacrylate, or tricalcium phosphate, or a combination thereof. In one embodiment, the one or more particulate materials of the putty A, the putty B, or both is present in an amount that is up to about 80 wt % of the final composition. In one embodiment, the one or more particulate material(s) is a carbonate or bicarbonate selected from calcium carbonate, magnesium carbonate, aluminum carbonate, iron carbonate, zinc carbonate, calcium bicarbonate, and sodium bicarbonate. In one embodiment, the one or more particulate materials do not comprise calcium carbonate or calcium phosphate. In one embodiment, the one or more particulate material(s) is selected from embedded particles of bone, demineralized bone, bone morphogenetic protein, hydroxyapatite, calcium phosphate, siliconized calcium phosphate, an inorganic material (e.g. stainless steel powder), a bone substitute material, a carbonate selected from magnesium carbonate, aluminum carbonate, iron carbonate, zinc carbonate, calcium carbonate, sodium carbonate, and a bicarbonate of magnesium, aluminum, iron, or zinc, or a combination of any of the previously listed options. In some embodiments of the composition of this disclosure, the putty A, the putty B, or both of the invention do not comprise a particulate material.

In some embodiments of the composition of this disclosure, the one or more additive material(s) of the putty A, the putty B, or both is a colorant, an antioxidant, an antibiotic, an antibacterial, an anti-infective, an active chemical hemostat, a steroid, calcium stearate, tocopheryl acetate, triacetin, a nonabsorbable plasticizer, other therapeutic agent(s), or any combination thereof. In some embodiments of the composition of this disclosure, the one or more additive material(s) of the putty A, the putty B, or both comprise a carbonate or bicarbonate selected from calcium carbonate, magnesium carbonate, aluminum carbonate, iron carbonate, zinc carbonate, calcium bicarbonate, sodium bicarbonate, embedded particles of bone, demineralized bone, bone morphogenetic protein, hydroxyapatite, calcium phosphate, siliconized calcium phosphate, aluminum carbonate, iron carbonate, zinc carbonate, calcium carbonate, sodium carbonate, and bicarbonate of magnesium, aluminum, iron, zinc, and combinations thereof. In some embodiments of the composition of this disclosure, the active chemical hemostat is a blood clot-inducing agent selected from a group consisting of prothrombin, thrombin, fibrinogen, fibrin, or any combination thereof. In some embodiments of the composition of this disclosure, the one or more additive material(s) of the putty A, the putty B, or both comprise starch, antimicrobial agent(s), colorant(s), x-ray opaque substance(s), and water. In some embodiments of the composition of this disclosure, the one or more additive material(s) of the putty A, the putty B, or both comprise a foaming agent (e.g., water). In some embodiments of the composition of this disclosure, the putty A, the putty B, or both do not comprise an additive material.

In some embodiments, the settable, nonabsorbable polyurethane, polyureaurethane, polyetherurethane or polyetherureaurethane compositions of the present disclosure are polymers or prepolymers formed from the reaction of (i) a nondegradable polyaromatic isocyanate, preferably a diisocyanate or a polyisocyanate, and (ii) a nondegradable polyol and/or a polyamine, with the optional addition of (iii) a chain extender or crosslinker or curative. The terms “isocyanate” and “polyisocyanate” as used throughout herein, may be used interchangeably to refer to the polyaromatic isocyanates used in making the curable, nondegradable polyurethane, polyureaurethane, polyetherurethane or polyetherureaurethane compositions of the invention. The term “polyisocyanate” encompasses a chemical structure having two or more isocyanate groups. The term “polyaromatic” refers to the isocyanate groups residing on two or more aromatic rings. The term “polyol” encompasses a chemical structure having two or more hydroxyl groups. As used herein, the term “polyol” refers to both diols and polyols. Similarly, the term “polyamine” refers to a chemical structure having two or more amino groups present, and can be used to refer to both diamines and polyamines.

The polyaromatic isocyanates used to form the polyurethane, polyureaurethane, polyetherurethane or polyetherureaurethane compositions of the invention comprise no hydrolysable linkages bridging the aromatic rings. The term “polyaromatic isocyanates” as used herein is meant to distinguish from aromatic isocyanates having only a single aromatic ring, such as toluene diisocyanate. The isocyanate, polyol, and chain extender components, as well as other optional components of the invention, are described in more detail below.

In some embodiments, the combination of the polyisocyanate component and the polyol and/or polyamine component results in an exothermic reaction that does not exceed about 60° C. and does not produce noxious fumes either during or after mixing. In some embodiments, the combination of the polyisocyanate component and the polyol and/or polyamine component results in an exothermic reaction that does not exceed 65° C. (e.g., ≤65° C., ≤62° C., ≤60° C., ≤ 55° C., ≤52° C., or ≤50° C.), and, preferably, remains below 50° C.

In some embodiments, the combination of the polyisocyanate component and the polyol and/or polyamine component disclosed herein, results in the formation of low-exotherm, biocompatible compositions that are suitable for use in vivo, in that their formation does not produce toxic fumes/compositions or heat that can cause damage to surrounding tissue. In some embodiments, the compositions of the present disclosure are nondegradable, and do not produce any toxic components that can leach into the surrounding tissue upon implantation of the invention, and elicits no adverse effects to the surrounding tissue.

In some embodiments of the composition of this disclosure, the compositions disclosed herein may further comprise a third, a fourth, a fifth (or more) putty compositions, and/or additional putty additives.

In some embodiments, the compositions of the present disclosure are osteoconductive. The term “osteoconductive” as used throughout herein, indicates that the compositions of the present disclosure supports the attachment and proliferation of osteocytes/osteoblasts. In some embodiments, the compositions of the present disclosure are nondegradable, but supports the attachment and growth of bone on its surface. In some embodiments, the compositions of the present disclosure are hemostatic compositions. The term hemostatic composition as used throughout herein, means that the compositions of the present disclosure are able to be applied to the surface of bleeding bone in its uncured/unset state, and are able to cease bone bleeding upon application to the bone tissue. In some embodiments, the bleeding is stopped immediately after application of the composition or within about 1 minute, or within 2-5 minutes, or within about 5-10 minutes. In some embodiments, the hemostatic compositions of the present disclosure are adhesive and capable of adhering to bone tissue. In some embodiments, the hemostasis caused by the compositions of the present disclosure is mechanical (tamponade). In some embodiments, the hemostasis caused by the compositions of the present disclosure is chemical. In some embodiments, the compositions of the present disclosure also contain one or more agents that act as active chemical hemostats. Non-limiting examples of the one or more agents that act as active chemical hemostats include blood clot-inducing agents such as prothrombin, thrombin, fibrinogen, or fibrin. In one embodiment, the composition may also comprise one or more of epinephrine, tannic acid, ferrous sulfate, and/or the double-sulfates of a trivalent metal and a univalent metal such as potassium aluminum sulfate and ammonium aluminum sulfate. In some embodiments, the composition of the present disclosure in either its fluid, putty, or solid form is also preferably mechanically and/or chemically hemostatic. The term “fluid form” as used throughout herein, refers to the uncured form of the composition which is a viscous fluid or putty, or which hardens or “cures” or “sets” into the solid form.

The present invention further provides self-setting (i.e., increased viscosity or hardening after mixing) compositions for medical use that are produced by mixing or kneading together the two or more individual reactive putties disclosed herein. In some embodiments, the individual constituent putties (e.g., putty A and putty B) are provided in sterile form, and are hand-mixed or mixed using a machine/apparatus, in situ prior to implantation. In some embodiments, the compositions formed by mixing the constituent putties disclosed herein, are capable of hardening in the body and are used for orthopedic applications as a bone hemostat, bone adhesive, bone void filler, or a bone cement or a combination thereof. The term “bone cement” as used throughout herein, is meant to distinguish certain embodiments of the invention from other embodiments, such as soft tissue adhesives, which may not possess mechanical properties suitable for use in bone repair. A bone cement composition of the invention when fully cured or set has a compressive strength, tensile strength, and elasticity suitable for use in bone repair or reconstruction. In some embodiments, the compositions of the present disclosure when cured or set (i.e., hardened or solidified), also bond to bone or metal surfaces and reach a self-supporting bond strength within about 90 minutes. In one embodiment, the cured or set composition of the present disclosure has a compressive strength of at least 30 to 150 MPa, a tensile strength of at least 20 to 80 MPa, and an elastic modulus of at least 1,400 to 1,800 MPa. In some embodiments, the cured, set, solid, or hardened form of the compositions of the present disclosure is sufficiently durable to be drillable or machinable. The mechanical properties of the cured, set, solid, or hardened form of the compositions of the present disclosure as outlined herein refer to the properties of the polyurethane, polyureaurethane, polyetherurethane or polyetherureaurethane alone, without the addition of other, optional materials. In some embodiments, the addition of other, optional materials further increases these physical properties (e.g., compressive strength). In some embodiments, the compositions of the present disclosure do not comprise an optional particulate material. In certain embodiments, the compositions of the present disclosure comprise one or more particulate material(s), in an amount of up to about 80% by weight of composition.

The use of the term “fully cured”, “fully set”, “solid”, or “hardened” form of the composition of the present disclosure is meant to distinguish this cured, set, solid, or hardened form from the viscous fluid or putty form of the constituent materials/components/putties that harden upon mixing or kneading. In some embodiments, the solid form of the compositions of the present disclosure bond to bone or metal surfaces, and reaches self-supporting bond strength within approximately 90 minutes. In some embodiments, the solid form of the compositions of the present disclosure possesses tensile and shear strength equal to natural bone within 72 hours of mixing the constituent materials. In some embodiments, the compositions of the present invention formed by mixing or kneading the constituent materials/components/putties harden into a solid form at room temperature or at body temperature in between 5 to 90 minutes.

The term “fluid form” of the compositions as used throughout herein, is a putty or viscous fluid that hardens (i.e., “cures” or “sets”) into the final solid form. The fluid form of the compositions of the present disclosure, is moldable or pliable and does not adhere appreciably to surgical gloves or instruments, but adheres well to moist bone surfaces. The fluid form of the compositions of the present disclosure, is resistant to dislodgement by surgical irrigation at the application site. The fluid form of the compositions of the present disclosure, is useful, for example, to fill a cavity in the bone, for injection through a syringe to the site of application, or for bone reconstruction. The fluid form of the compositions of the present disclosure remains in a moldable state at room temperature for up to 120 minutes. In some embodiments, the moldability time period of the compositions of the present disclosure varies from 5 to 120 minutes.

In some embodiments, the rate of curing of compositions of the present disclosure is increased, for example, by the addition of a catalyst as described in more detail herein. The aromatic polyisocyanate monomers described herein will react fastest with the polyamine component, then the polyol component, and react slowest with water. In some embodiments, the rate of cure of the compositions of the present disclosure is decreased, for example, by replacing primary polyols with secondary polyols in the composition, and similarly by replacing primary amines with secondary amines.

In some embodiments, during the curing of the polyurethane, polyureaurethane, polyetherurethane or polyetherureaurethane compositions of the present disclosure formed from constituent components (e.g., liquid or putty), the compositions may undergo a transition to a “taffy-like” state prior to becoming fully set, cured, solid, or hardened. The term “taffy” phase, used interchangeably with “putty-like” herein, is distinguished from the constituent putties described herein. In some embodiments, the constituent putties of the compositions of the present disclosure comprise particulate fillers to ensure their “putty-like” characteristics.

In some embodiments of the compositions of the present disclosure, the polyurethane, polyureaurethane, polyetherurethane or polyetherureaurethane compositions are nonabsorbable under physiological conditions. In some embodiments of the compositions of the present disclosure, the polyurethane, polyureaurethane, polyetherurethane or polyetherureaurethane compositions do not undergo hydrolytic degradation upon implantation in vivo. In some embodiments of the compositions of the present disclosure, the polyurethane, polyureaurethane, polyetherurethane or polyetherureaurethane compositions exhibit no clinically significant mass loss upon implantation as it relates to hydrolytic degradation. In some embodiments of the compositions of the present disclosure, the polyurethane, polyureaurethane, polyetherurethane or polyetherureaurethane compositions exhibit no clinically significant loss in mechanical properties, as a result of hydrolytic degradation, upon implantation in vivo. The term “mechanical properties” used herein include tensile, shear, and compressive strengths and moduli.

In some embodiments of the compositions of the present disclosure, the compositions of the present disclosure when fully cured or set, have a defined pore size. The porosity of the compositions of the present disclosure when fully cured or set is controlled through the inclusion of water, surfactants, and/or hydrolysable solid particles (e.g., sodium chloride or sucrose particles) during the process of combining the one or more polyisocyanate(s) with the one or more polyol(s) or polyamine(s) of the present disclosure, to form the polyurethane, polyureaurethane, polyetherurethane or polyetherureaurethane compositions of the present disclosure. In some embodiments, the porosity of the solid form of the polyurethane, polyureaurethane, polyetherurethane or polyetherureaurethane compositions of the present disclosure is controlled by the addition of water to a prepolymer containing polyisocyanate groups, wherein the water reacts with the isocyanate group to form carbon dioxide resulting in porosity. In some embodiments, the solid form of the polyurethane, polyureaurethane, polyetherurethane or polyetherureaurethane compositions of the present disclosure has an average pore size in the range of from about 5 to 700 microns. In some embodiments, the solid form of the polyurethane, polyureaurethane, polyetherurethane or polyetherureaurethane compositions of the present disclosure has an average pore size in the ranges from about 100 to 300 microns, from 200 to 500 microns, from 300 to 600 microns, and from 500 to 700 microns, or greater. In some embodiments, the solid form of the polyurethane, polyureaurethane, polyetherurethane or polyetherureaurethane compositions of the present disclosure has an average pore size in the submicron range. In some embodiments, the solid form of the polyurethane, polyureaurethane, polyetherurethane or polyetherureaurethane compositions of the present disclosure has an average pore sizes ranging from about 100 to 1000 nanometers, from 100 to 400 nanometers, from about 400 to 800 nanometers, from about 200 to 600 nanometers, or from about 500 to 900 nanometers. In some embodiments, porosity is introduced into the cured, set, hardened, or solid form of the polyurethane, polyureaurethane, polyetherurethane or polyetherureaurethane compositions of the present disclosure through the use of porous filler materials. Non-limiting examples of the porous filler materials can be any commercially available calcium phosphate with pore sizes of 200 microns or greater.

In some embodiments of the compositions of the disclosure, the putty A comprises a prepolymer formed from the reaction of excess polyisocyanate with the polyol/polyamine component. In some embodiments of the compositions of the disclosure, the putty B comprises a prepolymer formed from the reaction of excess polyol/polyamine component with the polyisocyanate component. In some embodiments of the compositions of the disclosure, the putty A comprises a prepolymer formed from the reaction of excess polyisocyanate with the polyol/polyamine component; and the putty B comprises a prepolymer formed from the reaction of excess polyol/polyamine component with the polyisocyanate component. In some embodiments of the compositions of the disclosure, the compositions of the invention described herein are formed by a process of combining a polyisocyanate prepolymer with a polyol or chain-extender and a catalyst, optionally with one or more particulate materials as described above, to form a poly(isocyanurate) composition, as shown below.

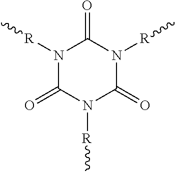

In another embodiment, the isocyanate prepolymer is combined with a polyol, water, and a catalyst, optionally with an osteoconductive filler, to form a poly(urethane-urea-isocyanurate) composition.

In some embodiments, the compositions of the present disclosure comprise an ether-linked polyaromatic triisocyanate and a trimethylolpropane ethoxylated polyol, wherein the compositions are formed from the reaction of the polyaromatic polyisocyanate, one or more polyols and/or polyamines, and, optionally, a polyol and/or a polyamine as a chain extender. In some embodiments, the composition further comprises one or more of water, a carboxylic acid (e.g., benzoic acid, as foaming agent), a divalent or polyvalent metal salt, a metal carbonate or bicarbonate, or a phosphate (e.g., for osteoconductivity). The ether-linked triisocyanate monomer as described herein has the following structure:

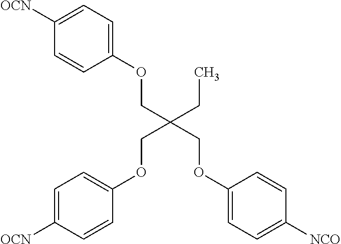

The trimethylolpropane ethoxylate polyol described herein has the following structure:

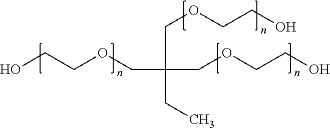

### The Polyisocyanate Component

The settable nonabsorbable compositions of the present disclosure can comprise constituent components (e.g., putties) comprising one or more di- or polyaromatic, di- or polyisocyanates containing no hydrolysable linkages within its structure. Hydrolysable polyurethanes can be fabricated utilizing a polyisocyanate that contains ester linkages. The polyisocyanate of the nonabsorbable compositions of the present disclosure contains only ether linkages, which are resistant to hydrolysis at physiological conditions.

In some embodiments, the nonabsorbable polyurethane invention described herein is prepared from one or more aromatic isocyanates selected from the following the compounds:

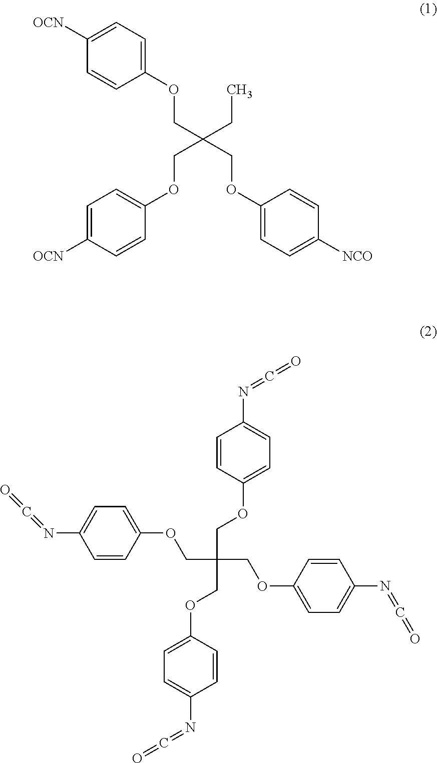

In a preferred embodiment of the compositions comprising a set of at least two reactive putties of the present disclosure, the constituent components (e.g., the first and at least the second putty or putty A or at least putty B) comprise any one or more of the di- or polyisocyanates disclosed herein. In certain embodiments, the polyisocyanate is an aromatic isocyanate, an aliphatic isocyanate, a cycloaliphatic isocyanate, or an adduct of an isocyanate.

### The Polyol/Polyamine Component

The settable nonabsorbable compositions of the present disclosure can comprise constituent components (e.g., putties) comprising one or more diols/polyols and/or diamines/polyamines suitable for use in forming the polyurethane, polyureaurethane, polyetherurethane or polyetherureaurethane compositions of the present disclosure, wherein the one or more diols/polyols and/or diamines/polyamines are nondegradable under physiological conditions.

The term “polyol” as used herein, refers to diols and polyols, unless indicated otherwise. In some embodiments, the compositions of the present disclosure are prepared by combining an excess of the polyisocyanate component with the polyol/polyamine component. In some embodiments, the relative amounts of the polyisocyanate component and the polyol/polyamine component are calculated as the molar ratio of NCO groups of the polyisocyanate component (I) to the active hydroxyl/amino functional groups (H) found in the polyol and polyamine. In some embodiments, the ratio of polyisocyanate to polyol/polyamine (I:H) is at least 2:1. In some embodiments, the ratio is about 1.5:1, about 3:1, or about 4:1. In some embodiments, the ratio is about 5:1, about 8:1, about 10:1, about 20:1, or about 50:1.

In some embodiments of the compositions of the present disclosure, the polyol/polyamine component is present in a polyisocyanate prepolymer in an amount ranging from about 5 wt % to about 50 wt % of the prepolymer. In some embodiments of the compositions of the present disclosure, the polyol/polyamine component is present in an amount ranging from about 5 wt % to about 10 wt %, about 10 wt % to about 20 wt %, about 20 wt % to about 35 wt %, about 25 wt % to about 40 wt %, or from about 35 wt % to about 50 wt % of the prepolymer.

Non-limiting examples of the one or more polyols suitable for use in the compositions of the present disclosure include biocompatible naturally occurring polyols, synthetic polyols, and mixtures thereof. In some embodiments, the one or more polyols of compositions of the present disclosure comprise at least one ether group. In some embodiments, the one or more polyols of compositions of the present disclosure comprise 2-4 ether groups or 5-10 ether groups. In some embodiments, the one or more polyols of compositions of the present disclosure comprise two or more hydroxyl groups. Non-limiting examples of the one or more polyols suitable for use in the compositions of the present disclosure include trimethylolpropane ethoxylate, triethanolamine, tetrakis(2-hydroxypropyl)ethylenediamine, tetrakis(2-hydroxyethyl)ethylenediamine, butanediol, poly(propylene glycol), isosorbide, or polycarbonate diols.

In some embodiments of the compositions of the present disclosure, a polyisocyanate prepolymer component is combined with a polyamine component to form a poly(urethane-urea). In some embodiments of the compositions of the present disclosure, the polyamine component is a primary or secondary diamine, or a hindered amine. Non-limiting examples of suitable polyamines of the compositions of the present disclosure comprise hindered diamine (e.g., isophorone diamine, or IPDA), 1,4-cyclohexyl diamine, 1,3-pentane diamine, or aliphatic secondary diamines, or a combination thereof. In some embodiments of the compositions of the present disclosure, the polyamine component comprises aliphatic diamines and/or cycloaliphatic diamines.

### The Polyol/Polyamine Component for Multi-Putty Compositions

Non-limiting examples of the one or more polyols suitable for use in the multi-putty (at least two putties) compositions of the present disclosure include any of the biocompatible, naturally occurring polyols, synthetic polyols, and mixtures thereof disclosed herein. In certain embodiments, the one or more polyols comprise at least one ether group. In certain embodiments, the one or more polyols comprise 2-4 ether groups or 5-10 ether groups. In certain embodiments, the one or more polyols have at least two hydroxyl groups. In certain embodiments, the one or more polyols have three or more hydroxyl groups.

The polyurethane, polyureaurethane, polyetherurethane or polyetherureaurethane reactants/components and reactions described herein, can be applied directly to the multi-putty compositions (at least two putties) of the present disclosure. In some embodiments, the compositions of the present disclosure are produced by mixing a first putty composition (e.g., “Component/Putty A”), which comprises one or more reactants capable of participating in chemical reactions with one or more reactants present in a second putty composition (e.g., “Component/Putty B”), and, optionally, with a reactive third, fourth, fifth or more reactants in any number of additional putties (e.g. any number as deemed necessary or useful to produce a product that is harder, less flowable, and/or more cohesive than the individual component putties). In some embodiments of the compositions of the present disclosure the individual components/putties can be formed by preparing a suspension of particulates within a liquid. In some embodiments of the compositions of the present disclosure the individual components/putties can comprise one or more moldable solids (e.g., a wax-like material, a particulate solid, or a modeling clay combined with a moldable solid and/or a moldable solid and a liquid).

The polyurethane, polyureaurethane, polyetherurethane or polyetherureaurethane compositions of the present disclosure can be formed by chemical reactions of two or more reactants (key reactive components) which produce a self-hardening or increased viscosity polymer, wherein the reactions are initiated when the two or more individual component putties comprising the key reactive components are mixed or combined. In some embodiments, the mixing of the two or more reactants (key reactive components) results in hardening and/or increased viscosity of the composition. In addition to the polyurethane reactions disclosed herein, non-limiting examples of other polymeric reactions include epoxy reactions and vinyl reactions. In some embodiments, the composition can be an epoxy adhesive or cement prepared by reacting a di-epoxide with an amine, such as a polyamine. In some embodiments, the composition can be a vinyl compound, such as methylmethacrylate, prepared by reacting molecules containing a vinyl or alkene group with benzoyl peroxide (radical induction) or ferric chloride (ionic induction).

In some embodiments of the polyurethane, polyureaurethane, polyetherurethane or polyetherureaurethane compositions of the present disclosure, the reactive components can be in particulate form. In some embodiments, the reactive components also provide a bulking feature to the putty. In some embodiments, the constituent components/putties comprise a vehicle, wherein the vehicle is non-reactive or comprises any material that does not detrimentally affect the reaction between the reactive components and/or the second putty. In some embodiments, the reactive components comprise liquid vehicles. In some embodiments, the reactive components are in liquid form, preferably a viscous fluid.

In some embodiments of the compositions of the present disclosure, the one or more of the individual components/putties are prepared in a suspension form. In some embodiments, in the components/putties in suspension form, particles are mixed with a liquid vehicle in proportions sufficient to produce a formable putty. In some embodiments, the particles of the suspension are less than 50, less than 40, less than 30, or less than 25 μm in diameter. In some embodiments, the particles of the suspension are less than 15 or less than 10 μm. In some embodiments, the particles of the suspension are nanoparticles. In some embodiments, the particles within the suspension are insoluble in the vehicle or the vehicle is saturated with a soluble form of the particulate phase, such that the particles themselves will not dissolve in the vehicle. In some embodiments, the components/putties of the composition disclosed herein comprise particles and liquid vehicles having similar hydrophilicity or hydrophobicity are used in ratios of up to 80:20 (wt:wt) of particle:vehicle. In some embodiments, the components/putties of the composition disclosed herein comprise 70, 60, or 50 wt % particles. In some embodiments, components/putties of the composition disclosed herein comprise equal to or less than 45 wt % particles. In some embodiments, the components/putties of the composition disclosed herein comprise reactive molecules within the individual components/putties present either in particulate or vehicle form.

In some embodiments, one or more of the constituents/putties of the compositions disclosed herein, can be prepared as a moldable solid. In some embodiments, the moldable solids have the moldability and texture of waxes, clays, or soft plastics. In some embodiments, the constituents/putties of the compositions disclosed herein, can comprise a softener (e.g., a nonreactive surfactant) to achieve the desired moldability and to allow adequate mixing with other component putties. In some embodiments of the compositions disclosed herein, the reactive components constituents/putties are prepared as a particulate solid. In some embodiments, the particulate solid is blended with a liquid or a wax-like formable material to produce the constituent putty. In some embodiments of the compositions disclosed herein, the reactive components in the constituents/putties are prepared as moldable solids. In some embodiments, the moldable solids are softened with a liquid vehicle.

The individual component/putty compositions of the compositions of the present disclosure can be formed by a process of combining the polyol and/or polyamine components, disclosed herein, and the polyisocyanate or the polyisocyanate prepolymer components, disclosed herein, to form the polyurethane, polyureaurethane, polyetherurethane or polyetherureaurethane compositions of the present disclosure. The combination of the polyol and/or polyamine components and the polyisocyanate or the polyisocyanate prepolymer components, results in a polymerization reaction that produces heat, but the incorporation of solids and/or fillers can serve as a heat sink to produce a modulated exotherm. The combination of the polyol and/or polyamine components and the polyisocyanate or the polyisocyanate prepolymer components does not produce/release any adverse fumes during or after mixing. In some embodiments, the polyol component is a biocompatible, naturally occurring or synthetic polyol, or a combination thereof, as described herein. In some embodiments, the polyisocyanate component is a nonhydrolyzable diisocyanate. In some embodiments, the process of combining the polyol and/or polyamine components disclosed herein further comprises combining the polyol/polyamine and polyisocyanate components with either water or carboxylic acid to form carbon dioxide, which makes the polymer porous.

In some embodiments of the composition of the present disclosure, wherein the one or more reactive components/putties are liquids or formable solids, the reactive components/putties are mixed with an additive in the form of a particulate filler to produce the components/putties. In addition to viscosity adjustment, additives can be added to the components/putties of the compositions of the present disclosure to affect specific features or properties of the final product compositions or the setting/cured polymer described herein. The specific features or properties of the final product compositions or the setting/cured polymer described herein, may include, but are not limited to, component putty softness and mixability, final product composition setting time, softness (e.g., moldability), polymerized product tissue adherence, prevention of adhesion formation, osteoconductivity, osteoinductivity, inflammation, absorption, drug delivery properties, and time, as well as other properties. In some embodiments, the reactive components of one or more components/putties of the compositions of the present disclosure are pre-reacted to produce a polymerized or a partially polymerized product. In some embodiments, the polymerized or partially polymerized product is reduced to particulate form through standard methods such as cryomilling. In some embodiments, the particles of the pre-reacted polymerized product can then be used as all, or a portion of, particulate material.

In some embodiments, the additive material(s) described herein can be added in the role of a filler to produce a constituent component/putty of the compositions of the present disclosure. In some embodiments, the additive material is a calcium salt, wherein the composition is preferably used for bone applications. In some embodiments, the calcium salt is any one of calcium salts of fatty acids, phospholipids, calcium carbonate, calcium sulfates, or calcium phosphates, or a combination thereof. In some embodiments, the additive material(s) can be other fillers. In some embodiments, the other fillers cane be any of ceramics, glasses, bioglasses, cholesterols, or binders, or a combination thereof. In some embodiments, the reactive components for the constituents/putties of the compositions of the present disclosure, can be present in the form of moldable solids. In some embodiments, the moldable solids can be waxes composed of fatty alcohol esters or polymers, such as polyethylene glycol.

In some embodiments of the compositions of the present disclosure, the process of combining the polyol and/or polyamine components disclosed herein can comprise the inclusion of an osteoconductive additive, such as carbonate (e.g., calcium carbonate, magnesium carbonate, aluminum carbonate, iron carbonate, zinc carbonate, calcium bicarbonate, and sodium bicarbonate). Nonlimiting examples of osteoconductive materials include ceramics, such as substituted calcium phosphates (e.g., silicate, strontium, or magnesium substituted) and glasses, such as bioglass. In some embodiments, the process of combining the polyol and/or polyamine components disclosed herein comprise the inclusion of a surfactant, as least one radiopaque substance, or at least one protein, or a combination thereof. The process of combining the polyol and/or polyamine components disclosed herein further comprise inclusion of a crosslinker. In some embodiments, the crosslinker is a trifunctional castor oil-based polyol. In some embodiments, the process of combining the polyol and/or polyamine components disclosed herein further comprises the inclusion of one or more of the following: bone, demineralized bone matrix, bone morphogenetic protein, calcium phosphate, siliconized calcium phosphate, calcium pyrophosphate, hydroxyapatite, poly(methyl methacrylate), glass-ionomer, calcium sulfate, tricalcium phosphate, bone-like mineral (e.g., crystalline hydroxyapatite or calcium pyrophosphate).

In some embodiments, the compositions of the present disclosure are formed by a process of combining a polyisocyanate prepolymer with a polyol, chain extender, or catalyst, or optionally with an osteoconductive filler to form a poly(urethane-isocyanurate) composition. In some embodiments, the isocyanate prepolymer is combined with a polyol, water, and a catalyst, or optionally with an osteoconductive filler to form a poly(urethane-urea-isocyanurate) composition.

### Osteoconductive Additives and Filler Materials

Non-limiting examples of osteoconductive additives that can be included in the compositions of the present disclosure include carbonate (e.g., calcium carbonate, magnesium carbonate, aluminum carbonate, iron carbonate, zinc carbonate, calcium bicarbonate, and sodium bicarbonate), bone (e.g., demineralized bone matrix, bone morphogenetic protein, allograft bone, and/or autologous bone), calcium phosphate, siliconized calcium phosphate, substituted calcium phosphates (e.g., with magnesium, strontium, or silicate), calcium pyrophosphate glass, calcium sulfate, tricalcium phosphate (e.g., β-tricalcium phosphate), or any combination thereof.

In some embodiments, the one or more constituent components/putties of the compositions of the present disclosure comprise an optional additive material in an amount of from about 0.01 wt % to about 80 wt % of the composition. In some embodiments, the one or more constituent components/putties of the compositions of the present disclosure comprise the additive material in an amount of 5-10 wt %, 10-20 wt %, 25-35 wt %, 20-40 wt %, 35-55 wt %, 50-70 wt %, 65-80 wt %, or more than 80 wt % of the composition. In some embodiments, the optional additive is in nano-scale sizes. In some embodiments, the optional additive is in micron or millimeter sizes, or mixtures thereof.

### Other Optional Additives

In some embodiments, the compositions of the present disclosure comprise one or more “cell openers”. Non-limiting examples of cell openers as described herein include ORTOGEL501 (Goldschmidt) and X-AIR (Specialty Polymers & Services). In some embodiments, the cell openers are present in an amount from about 0.1 wt % to about 5 wt % of the composition. In some embodiments, the cell openers are present in amounts of about 1 wt % to about 2 wt %. In some embodiments, the cell openers are present in amounts of about 1 wt % to about 3 wt % of the composition.

In some embodiments, the compositions of the present disclosure comprise one or more antibiotics. Non-limiting examples of antibiotics as described herein include beta-lactam antibiotics such as tobramycin, subclasses Penicillins (examples: penicillin G, methicillin, oxacillin, ampicillin, amoxicillin), Cephalosporins, Glycopeptides (example vancomycin), Carbapenems (examples imipenem and meropenem), Polymyxin and Bacitracins (example bacitracin, neomycin) or Lipopeptides (example daptomycin), Protein synthesis inhibitors such as subclasses Aminoglycosides (example gentamicin, streptomycin, kanamycin), Tetracyclines (examples tetracycline, doxycycline, minocycline, and tigecycline), Oxazilodinone (linezolid), Peptidyl transferases (example Chloramphenicol), Macrolides (examples erythromycin, azithromycin, telithromycin), Lincosamides (examples clindamycin), and Streptogramins (example prisintamycin), DNA synthesis inhibitors such as metronidazole and subclass Fluoroquinolones (examples ciprofloxacin, norfloxacin, morifloxacin), RNA synthesis inhibitors such as rifampin, Mycolic acid synthesis inhibitors such as isoniazid, and Folic acid synthesis inhibitors such as Trimethoprim and subclass Sulfonamides (examples sulfamethoxazole, sulfadoxin). In some embodiments, the antibiotic is present in an amount ranging from about 0.01 wt % to about 8 wt % of the composition. In certain embodiments, gentamicin is present at a concentration from about 10 mg/cc to about 200 mg/cc of the composition. In certain embodiments, vancomycin is present from about 40 mg/cc to about 600 mg/cc of the composition. In certain embodiments, minocycline is present from about 5 mg/cc to about 200 mg/cc of the composition. In certain embodiments, rifampin is present from about 10 mg/cc to about 300 mg/cc of the composition.

In some embodiments, the compositions of the present disclosure comprise one or more local anesthetics. Non-limiting examples of local anesthetics include lidocaine, bupivacaine, tetracaine, and ropivacaine, including the freebases, their salts, and derivatives thereof.

In some embodiments, the compositions of the present disclosure comprise one or more antioxidants. Non-limiting examples of suitable antioxidants include Vitamin E acetate, IRGANOX 1010 and IRGANOX 1035 (Ciba Geigy), and CYANOX 1790 and CYANOX 2777 (Cytec Industries). In some embodiments, the antioxidant is present in an amount ranging from about 0.01 wt % to about 5 wt % of the composition.

In some embodiments, the compositions of the present disclosure comprise a steroid-based compound, such as an intracellular messenger, to modulate the rate of bone growth. In some embodiments, the compositions of the present disclosure comprise progenitor cells.

### Constituent Putty Viscosity

In some embodiments, the putty-like consistency of the compositions or the constituent components/putties of the present disclosure is achieved by appropriate adjustment of the liquid-to-solid ratio. In some embodiments, the putty-like consistency of the compositions or the constituent components/putties of the present disclosure is achieved by varying particle size, wherein smaller particles result in smoother, more cohesive putties. In some embodiments the reactive components of the compositions or the constituent components/putties of the present disclosure, which are liquids and/or powders are alternatively, or additionally, partially reacted by limiting one or more of the reactants to produce more viscous versions of the liquid components. In some embodiments, the reactive components of the compositions or the constituent components/putties of the present disclosure can comprise softeners, such as nonreactive surfactants, hydrophobic polymers, mineral oil, triacetin, etc.

In some embodiments, wherein the reactive components of the one or more constituent components/putties of the compositions of the present disclosure are particulate solids, the reactive components are mixed with a liquid or moldable solid in order to produce a useful putty. In some embodiments, the viscosity of the compositions or the constituent components/putties of the present disclosure is further adjusted. In some embodiments, the further adjusting of the viscosity is done by adding additional liquid. In some embodiments, the compositions or the constituent components/putties of the present disclosure comprise vehicles to affect specific features of the constituent putties, final product composition, or the setting/cured polymer. Non-limiting examples of the properties include component putty softness and mixability, final product composition setting time or softness (e.g., moldability), polymerized product tissue adherence, prevention of tissue adhesion, formation, osteoconductivity, osteoinductivity, inflammation, absorption, drug delivery properties, and time, among others.

### Water

In some embodiments, the compositions of the present disclosure contain no added water. In some embodiments, the compositions of the present disclosure are anhydrous. In some embodiments of the compositions of the present disclosure, wherein there is no added water in the compositions, water may nevertheless be present in small amounts. In some embodiments of the compositions of the present disclosure, the compositions of the present disclosure comprise certain commercially-available polyols, wherein commercially-available polyols comprise a mixture of the polyol and a small amount of water. In some embodiments of the compositions of the present disclosure, alternately or additionally, the particulate materials as described herein, such as calcium carbonate, can comprise water bound within. In some embodiments, the compositions of the present disclosure are formulated in an atmosphere that contains moisture resulting in the incorporation of water into the compositions. In some embodiments, the compositions of the present disclosure are prepared under a nitrogen purge that comprises a desired amount of moisture, thereby controlling the water content of the compositions. In other embodiments, water may be added to the compositions during the process of their formation from the component parts. In other embodiments, the compositions are prepared under essentially water-free conditions with anhydrous components such that the resulting compositions are essentially anhydrous.

In some embodiments of the compositions of the present disclosure water is present in the compositions being made in an amount from at least 0.01 wt % to 3 wt % of the composition. In certain embodiments, water is present in an amount ranging from 0.05 wt % to 1 wt %, from 0.05 wt % to 1.5 wt %, from 0.1 wt % to 1 wt %, from 0.1 wt % to 1.5 wt %, from 0.1 wt % to 2 wt %, from 1 wt % to 2 wt %, or from 2 wt % to 3 wt %.

### Particulate Materials

Both the putty and non-putty compositions of the invention may contain optional particulate materials. In one embodiment, the particulate material is an osteoconductive material. In certain embodiments, the particulate material supports cell attachment at the application site. In certain embodiments, the mean particle size of the optional particulate material is in the micron or submicron range. In one embodiment, the mean particle size is from 0.001 to 0.100 μm, from 0.100 to 1 μm, from 1 to 5 μm, from 5 to 500 μm, or from 500 to 1000 μm.

In some embodiments, the particulate material is a carbonate or bicarbonate (e.g., calcium carbonate, magnesium carbonate, aluminum carbonate, iron carbonate, zinc carbonate, calcium bicarbonate, sodium bicarbonate, or any combination thereof). In some embodiments, the particulate material is bone (e.g., demineralized bone matrix, bone morphogenetic protein, allograft bone, and/or autologous bone), calcium phosphate, siliconized calcium phosphate, substituted calcium phosphates (e.g., with magnesium, strontium, or silicate), calcium pyrophosphate, hydroxyapatite, poly(methyl methacrylate), glass-ionomer, calcium sulfate, tricalcium phosphate (e.g., β-tricalcium phosphate), or any combination thereof. In some embodiments, the particulate material is a polyether ether ketone (PEEK), REPLACE (Cortek, Inc.), and EX-PANCEL (Akzo Nobel). In some embodiments, the particulate material is a ceramic, such as substituted calcium phosphates (e.g., silicate, strontium, or magnesium substitution), or a glass, such as bioglass. In some embodiments, the particulate material is one or more of calcium sulfate, calcium phosphosilicate, sodium phosphate, calcium aluminate, calcium phosphate, hydroxyapatite, demineralized bone matrix, or mineralized bone. In some embodiments, the particulate material, when present, may comprise any one or more of the materials listed in the embodiments above. In one embodiment, the particulate material, if present in the composition, does not comprise calcium carbonate.

In some embodiments, the compositions or the constituent components/putties of the compositions of the present disclosure comprise the particulate material in an amount ranging from 0.01 to 10 wt % of the composition. In some embodiments, the compositions or the constituent components/putties of the compositions of the present disclosure, comprise the particulate material in an amount ranging from 0.10 to 10 wt %, 1 to 10 wt %, or 5 to 10 wt %. In some embodiments, the compositions or the constituent components/putties of the compositions of the present disclosure comprise the particulate material in an amount ranging from 10 to 20 wt %, 20 to 30 wt %, 30 to 40 wt %, 40 to 50 wt %, 50 to 60 wt %, 60 to 70 wt %, or 70 to 80 wt % of the composition.

### Foaming Agents

In some embodiments, the compositions or the constituent components/putties of the compositions of the present disclosure comprise an optional foaming agent to, for example, modulate pore size. In some embodiments, the foaming agent is carboxylic acids, wherein the carboxylic acids react with isocyanates present to form carbon dioxide (and the corresponding amide). Non-limiting examples of carboxylic acids that can be used in this manner are benzoic acid, malic acid, and succinic acid. In certain embodiments, some embodiments, the compositions or the constituent components/putties of the compositions of the present disclosure are formed by a process of combining a polyol and/or polyamine, polyisocyanate, and a carboxylic acid. In some embodiments, the carboxylic acid does not contain water. In some embodiments, the compositions or the constituent components/putties of the compositions of the present disclosure formed with carboxylic acid do not contain added water. In some embodiments, the compositions or the constituent components/putties of the compositions of the present disclosure comprise albumen as a foaming agent, with or without sodium alginate. In another embodiment, the compositions or the constituent components/putties of the compositions of the present disclosure comprise hydrogen peroxide as a foaming agent.

### Catalyst Component

In some embodiments, the compositions or the constituent components/putties of the compositions of the present disclosure comprise an optional catalyst (e.g., added to the polyol that is combined with the isocyanate to form the compositions of the invention). In certain embodiments, the compositions or the constituent components/putties of the compositions of the present disclosure comprise at least one catalyst in an amount sufficient to ensure that the polymerization reactions have proceeded to completion before the compositions are placed within the body of a subject. Non-limiting examples of catalysts include a tertiary amine (e.g., DABCO 33LV, Air Products, Inc.) and organometallic compounds, such as stannous octoate and dibutyl tin dilaurate. In some embodiments, the catalyst may remain in the compositions of the disclosure, after its formulation and curing, for example, as a monomer that is present in the matrix of the solidified form of the composition. A non-limiting example of such a catalyst is N,N,N′-Tri (2-hydroxylpropyl)-N′-hydroxyethyl ethylene diamine (POLY-Q-40-800, Arch Chemicals, Inc.).

In some embodiments, the compositions or the constituent components/putties of the compositions of the present disclosure comprise a catalyst in an amount ranging from about 0.05 wt % to about 0.5 wt % of the polyol. In some embodiments, the compositions or the constituent components/putties of the compositions of the present disclosure comprise a catalyst in an amount ranging from about 0.15 wt % to about 0.4 wt % of the polyol.

### Optional Surfactant Component

In some embodiments, the compositions or the constituent components/putties of the compositions of the present disclosure comprise an optional surfactant in order to control the porosity of the composition including the size and/or shape of the pores within the composition. Non-limiting examples of suitable surfactants include DABCO DC 193 and DABCO DC 5241 (Air Products, Inc.), MAXEMUL 6106 (Uniqema), and silicone surfactants (e.g., those available from Struktol Corp.).

### Radiotransparent/Radiopaque Component

In some embodiments, the compositions or the constituent components/putties of the compositions of the present disclosure comprise an optional filler, wherein the filler is radiopaque (e.g., calcium phosphate granules) and imparts radiopacity to the hardened composition. In some embodiments, the compositions or the constituent components/putties of the compositions of the present disclosure comprise an optional radiotransparent and/or a radiopaque substance. Non-limiting examples of a radiotransparent substance include air, nitrogen gas, carbon dioxide, and oxygen gas. Non-limiting examples of a radiopaque substance include ceramic particles (e.g., calcium phosphate), barium sulfate (BaSO₄), and zirconium dioxide (ZrO₂). Examples of commercially available radiopaque substances include LIPIODOL, HYPAQUE, and OMNIPAQUE. In some embodiments, the compositions or the constituent components/putties of the compositions of the present disclosure comprise a radiotransparent and/or radiopaque substances in amounts of about 5 wt % to about 30 wt % of the composition, and, in certain embodiments, from about 10 wt % to about 20 wt % of the composition.

### Protein Component

In some embodiments, the compositions or the constituent components/putties of the compositions of the present disclosure optionally comprise one or more bioactive proteins, pep-tides, or polypeptides. In some embodiments, the one or more bioactive proteins, peptides, or polypeptides is active in the stimulation of bone growth. Non-limiting examples of suitable proteins include collagen, OP1 (Stryker Homedica), INFUSE (Medtronic Corp.), or any recombinant bone morphogenic protein. In some embodiments, the one or more bioactive proteins, peptides, or polypeptides is non-reactive with the other components of the composition, allowing it to be included at any point during the formulating process. In some embodiments, the one or more peptides is not incorporated into the polymer backbone of the compositions or the constituent components/putties of the compositions of the present disclosure, but instead is either embedded in the polymer matrix, dispersed in the composition, or adherent to the surface of the compositions or the constituent components/putties of the compositions.

The one or more bioactive proteins, peptides, or polypeptides may be incorporated within the compositions or the constituent components/putties of the compositions of the present disclosure, for example, by inclusion in the process of combining the polyisocyanate component and the polyol/polyamine component. In some embodiments of the compositions or the constituent components/putties of the compositions of the present disclosure, the one or more bioactive proteins, peptides, or polypeptides is dispersed throughout the composition or the constituent components/putties of the compositions. In some embodiments of the compositions or the constituent components/putties of the compositions of the present disclosure, the one or more bioactive proteins, peptides, or polypeptides is added after combination of the other components. In some embodiments of the compositions or the constituent components/putties of the compositions of the present disclosure, the one or more bioactive proteins, peptides, or polypeptides is added about 10 to 45 minutes after combination of the other components. In some embodiments, the one or more bioactive proteins, peptides, or polypeptides adheres to an outer surface of the composition.

### Optional Light- or Photo-Initiators

In some embodiments, the compositions or the constituent components/putties of the compositions of the present disclosure comprise light- or photo-initiators. Non-limiting examples of suitable light- or photo-initiators include 24650-42-8 (Loctite Corp.). In a preferred embodiment, the light- or photo-initiators are included in compositions or the constituent components/putties of the compositions of the present disclosure, made from unsaturated components (e.g., isocyanate prepolymers having one or more double bonds or polyols having polarized double bonds). In some embodiments, a photo- or light-initiator is incorporated into the compositions or the constituent components/putties of the compositions of the present disclosure, by combining with a liquid component (e.g., a polyisocyanate, a polyol, a polyamine, a chain extender, or a crosslinker). In certain embodiments, the compositions or the constituent components/putties of the compositions of the present disclosure, comprising a photo- or light-initiator, solidify at an accelerated rate (e.g., in the range of about 1 to 5 minutes or 1 to 10 minutes) after exposure to a suitable energy source (e.g., a suitable light source).

### Other Optional Additives

In certain embodiments, the compositions or the constituent components/putties of the compositions of the present disclosure optionally comprise one or more “cell openers”. Non-limiting examples of cell openers include ORTOGEL501 (Goldschmidt) and X-AIR (Specialty Polymer & Services). In certain embodiments, the compositions or the constituent components/putties of the compositions of the present disclosure comprise one or more cell openers in an amount ranging from about 0.1 wt % to about 5 wt % of the composition. In some embodiments, the compositions or the constituent components/putties of the compositions of the present disclosure comprise one or more cell openers in an amount ranging from about 1 wt % to about 2 wt % or about 1 wt % to about 3 wt % of the composition.

In some embodiments, the compositions or the constituent components/putties of the compositions of the present disclosure optionally comprise one or more antibiotics. Non-limiting examples of antibiotics as described herein include beta-lactam antibiotics such as tobramycin, subclasses Penicillins (examples: penicillin G, methicillin, oxacillin, ampicillin, amoxicillin), Cephalosporins, Glycopeptides (example vancomycin), Carbapenems (examples imipenem and meropenem), Polymyxin and Bacitracins (example bacitracin, neomycin) or Lipopeptides (example daptomycin), Protein synthesis inhibitors such as subclasses Aminoglycosides (example gentamicin, streptomycin, kanamycin), Tetracyclines (examples tetracycline, doxycycline, minocycline, and tigecycline), Oxazilodinone (linezolid), Peptidyl transferases (example Chloramphenicol), Macrolides (examples erythromycin, azithromycin, telithromycin), Lincosamides (examples clindamycin), and Streptogramins (example prisintamycin), DNA synthesis inhibitors such as metronidazole and subclass Fluoroquinolones (examples ciprofloxacin, norfloxacin, morifloxacin), RNA synthesis inhibitors such as rifampin, Mycolic acid synthesis inhibitors such as isoniazid, and Folic acid synthesis inhibitors such as Trimethoprim and subclass Sulfonamides (examples sulfamethoxazole, sulfadoxin). In some embodiments, the antibiotic is present in an amount ranging from about 0.01 wt % to about 8 wt % of the composition. In certain embodiments, gentamicin is present at a concentration from about 10 mg/cc to about 200 mg/cc of the composition. In certain embodiments, vancomycin is present from about 40 mg/cc to about 600 mg/cc of the composition. In certain embodiments, minocycline is present from about 5 mg/cc to about 200 mg/cc of the composition. In certain embodiments, rifampin is present from about 10 mg/cc to about 300 mg/cc of the composition.

In some embodiments, the compositions or the constituent components/putties of the compositions of the present disclosure optionally comprise one or more local anesthetics or analgesics. Non-limiting examples of local anesthetics or analgesics include lidocaine, bupivacaine, tetracaine, and ropivacaine. Further non-limiting examples of local anesthetics or analgesics include benzocaine and fentanyl (a potent synthetic opioid).

In some embodiments, the compositions or the constituent components/putties of the compositions of the present disclosure optionally comprise one or more anti-inflammatory substances, such as the nonspecific ibuprofen and/or aspirin, or the COX-2 specific inhibitors, such as rofecoxib and celeboxib.

In some embodiments, the compositions or the constituent components/putties of the compositions of the present disclosure optionally comprise one or more antioxidants. Non-limiting examples of suitable antioxidants include IRGANOX 1010 and IRGANOX 1035 (Ciba Geigy), and CY-ANOX 1790 and CYANOX 2777 (Cytec Industries). In some embodiments, the compositions or the constituent components/putties of the compositions of the present disclosure comprise one or more antioxidants in an amount ranging from about 0.01 wt % to about 0.5 wt % of the composition.

In some embodiments the compositions or the constituent components/putties of the compositions of the present disclosure further comprise a colorant. Non-limiting examples of suitable colorants include gentian violet, D&C Violet #2, and D&C Green #6.

In some embodiments, the compositions or the constituent components/putties of the compositions of the present disclosure comprise a steroid-based compound. Such an intracellular messenger may optionally be included in the compositions of the invention to modulate the rate of bone growth. In some embodiments, progenitor cells optionally may be included in the compositions of the invention.

### Clinical Applications

In some embodiments of the compositions of the present disclosure, the constituent components/putties can be mixed to relative homogeneity by hand or with a mixing apparatus, such as a mortar and pestle, to produce the compositions (final product compositions) as described herein. Depending upon the specific reaction being employed, the final product composition will begin to harden over time. During this phase, the compositions may be applied to bleeding bone to act as a hemostatic tamponade. In other embodiments, the compositions of the present disclosure can be applied as an adhesive (e.g., to stabilize a bone fracture or re-approximate a sternotomy). In other embodiments, the compositions of the present disclosure can be applied as a bone cement to fill gaps in the skeletal system, resulting in skeletal fusion, or aid in the adhesion between bone segments, fragments, and/or metallic hardware. In other embodiments, the compositions of the present disclosure can be custom shaped by a clinician to create form-fitting fixation devices such as sheets, rods, wraps, or other support structures that can be anchored by plates, sutures, or screws.

### Hardened Polymer

A hardened polymer, preferably containing an osteoconductive filler, may be ground to a fine powder and used as such, or converted into a putty, by mixing with a suitable vehicle, to fill bone voids and other orthopedic defects. The constituent putty could be used during manufacturing as an alternative to “conventional” polymerization using liquids and fillers to form fully cured materials due to the improved handling properties that eliminate liquid and taffy phases of polymerization.

The present disclosure also provides intraoperative use of the settable, nonabsorbable compositions of the present disclosure for tissue repair and/or reconstruction. The settable, nonabsorbable compositions of the present disclosure can be used for repair and/or reconstitution of two or more pieces of bone, two or more pieces of cartilage, and/or two or more pieces of bone and cartilage.

The present disclosure also provides a method of stabilizing, repairing, or reapproximating a bone fracture or a sternotomy, the method comprising the steps of: a) intraoperatively mixing or kneading together a set of two or more reactive putties, putty A and at least a putty B of any one of the compositions of the present disclosure, to form a moldable, settable, nonabsorbable polyurethane, polyureaurethane, polyetherurethane or polyetherureaurethane composition (nonabsorbable composition) at room or body temperature; b) applying the mixed or kneaded composition to the surfaces of the bone fracture or the cut surfaces of the sternotomy and manually reducing or reapproximating the bone fragments while allowing the composition to set; and c) allowing the composition to harden into its fully cured, solid form.

In some embodiments of the method of stabilizing, repairing, or reapproximating a bone fracture or a sternotomy disclosed herein, step (b) further comprises applying a portion of the composition across the surface of a surgical hardware to create a composition-hardware construct, and affixing the composition-hardware construct to the surfaces of the bone fracture or the cut surfaces of the sternotomy.

In some embodiments, the intraoperatively mixing or kneading together of a set of two or more reactive putties, putty A and at least putty B, of any one of the compositions of the present disclosure is done by any one of hand mixing or kneading or by using a mixing or kneading apparatus. In some embodiments, the method comprises a single step of applying a curable polyurethane, polyureaurethane, polyetherurethane or polyetherureaurethane composition of the invention to the bone fracture or the cut surfaces, with or without a catalyst. In some embodiments, the method comprises mixing the putty A with putty B to form the settable, nonabsorbable polyurethane, polyureaurethane, polyetherurethane or polyetherureaurethane composition, just prior to the application of said composition to the bone fracture or the cut surfaces, with or without a catalyst.

In certain embodiments, the method for stabilizing, repairing, or reapproximating of bone fragments of the present disclosure comprises intraoperatively mixing or kneading together a set of at least two reactive putties, putty A and at least putty B, of the composition disclosed herein to form the nonabsorbable composition disclosed herein, optionally dividing the nonabsorbable composition into two or more additional portions, applying a first portion of the composition to the cut or fractured surface of at least one of the pieces of bone, and maintaining the pieces of cut or fractured bone in proximity to form a reduced fracture until the nonabsorbable composition has hardened and the reduced fracture remains fixed. In certain embodiments, the first portion of the nonabsorbable composition is applied to the cut or fractured surface of at least one of the pieces of bone, either in multiple portions at a plurality of locations of the cut or fractured surface, and interrupted by gaps, or as a single portion across substantially the entire length of the cut or fractured surface. In embodiments, the method further comprises compressing the pieces of cut or fractured bone together until the first portion of the nonabsorbable composition has hardened. In certain embodiments, the pieces of cut or fractured bone are maintained in proximity for about 2 to 5 minutes. In certain embodiments, the method further comprises applying a second portion of the nonabsorbable composition disclosed herein across the reduced fracture line in the form of a plate or tape. In certain embodiments, the method further comprises pressing additional portions of the nonabsorbable composition into each of two or more drill holes located opposite each other across the reduced fracture line, thereby substantially filling each drill hole. In certain embodiments, the method further comprises shaping an additional portion of the nonabsorbable composition into a rod and joining each end of the rod to a portion of nonabsorbable composition pressed into a drill hole. In certain embodiments, the method further comprises drilling a hole into the nonabsorbable composition which is in the form of a plate or tape after the nonabsorbable composition has hardened.

In some embodiments of the method for stabilizing, repairing, or reapproximating of bone fragments of the present disclosure, the bone is a long bone, a short bone, a flat bone, an irregular bone, or a sesamoid bone. In some embodiments, wherein the bone is any one of a long bone or a short bone, the method further comprises stretching an additional portion of the nonabsorbable composition into the form of a ribbon or cuff and wrapping the nonabsorbable composition ribbon or cuff around the circumference of the reduced fracture line. In some embodiments, wherein the bone is a flat bone, the flat bone can be selected from a sternum, rib, scapula, pelvic bone or cranial bone. In embodiments where the bone is flat bone, the flat bone can be selected from a rib, scapula, or cranial bone. In some embodiments, wherein the bone is a rib bone, the method further comprises applying an additional portion of the nonabsorbable composition into the hollow of the rib bone.

In some embodiments, the bone is an irregular bone. In some embodiments, the irregular bone is a vertebra. In some embodiments, wherein the bone is a vertebra, the method further comprises inserting an additional portion of the nonabsorbable composition into an intervertebral space to form a spacer or cage. In some embodiments, the method further comprises applying a second portion of the nonabsorbable composition to two or more spinal pedicles adjacent to the nonabsorbable composition spacer or cage to form two or more nonabsorbable composition anchor points on the pedicles and either stretching a further portion of nonabsorbable composition between the anchor points or positioning a rod between the anchor points and pressing the rod into the anchor points, thereby connecting the anchor points.

In some embodiments, the method for stabilizing, repairing or reapproximating of bone fragments of the present disclosure comprises joining at least two pieces (e.g., 2, 3, 4, 5, 6, 7, 8, 9, or more) of fractured long or short bone, the method comprising intraoperatively mixing together two or more individual reactive putties to form a nonabsorbable composition, dividing the nonabsorbable composition into at least two portions, applying a first portion of the nonabsorbable composition to the cut or fractured surface of at least one of the pieces of bone, maintaining the at least two pieces of cut or fractured bone in proximity to form a reduced fraction until the nonabsorbable composition has hardened sufficiently to maintain the reduced fracture repair fixed, shaping a second portion of nonabsorbable composition into the form of a ribbon or cuff and wrapping the nonabsorbable composition ribbon or cuff around the circumference of the reduced fracture line, optionally intraoperatively mixing two or more additional individual reactive putties to form one or more additional nonabsorbable composition(s) and using the one or more additional nonabsorbable composition(s) in one or more of the following further optional step(s): shaping the one or more additional nonabsorbable composition(s) into one or more additional ribbon(s) or cuff(s) and wrapping the one or more additional ribbon(s) or cuff(s) around the circumference of the reduced fracture line, pressing portions of the one or more additional nonabsorbable composition(s) into each of two or more drill holes located opposite each other across the reduced fracture line, thereby substantially filling each drill hole, and/or shaping a portion of the one or more additional nonabsorbable composition(s) into a rod and joining each end of the rod to a portion of nonabsorbable composition pressed into a drill hole (e.g., by bending each end of the rod in order to fit it into a drill hole). In some embodiments, the methods of the present disclosure can be used to join shattered pieces of bone.

In some embodiments, the method for stabilizing, repairing, or reapproximating of bone fragments of the present disclosure comprises intraoperatively mixing two or more individual reactive putties to form a nonabsorbable composition, forming the nonabsorbable composition into the form of a ribbon or cuff, and wrapping the nonabsorbable composition ribbon or cuff around a circumference of the reapproximated long or short bone, thereby applying an internal orthopedic cast to a reapproximated long or short bone.

In embodiments, the method for stabilizing, repairing or reapproximating of bone fragments of the present disclosure, the bone fracture is of one or more vertebra (e). In some embodiments, the method comprises intraoperatively mixing two or more individual reactive putties to form a nonabsorbable composition, optionally dividing the nonabsorbable composition into two or more additional portions, and inserting a first portion of the nonabsorbable composition into an intervertebral space to form a spacer or cage. In some embodiments, the method further comprises introducing one or more of an autograft material, an allograft material, and/or a bone substitute material into one or more hole(s) drilled into the nonabsorbable composition spacer or cage. In some embodiments, the method further comprises applying a second portion of the nonabsorbable composition to two or more spinal pedicles adjacent to the nonabsorbable composition spacer or cage to form two or more of the nonabsorbable composition anchor points on the pedicles and either stretching a further portion of the nonabsorbable composition between the anchor points or positioning a rod between the anchor points and pressing the rod into the anchor points, thereby connecting the anchor points.

In some embodiments, the method for stabilizing, repairing, or reapproximating of bone fragments of the present disclosure comprises intraoperatively mixing two or more individual reactive putties to form a nonabsorbable composition of the present disclosure, optionally dividing the nonabsorbable composition into two or more additional portions, applying a first portion of the nonabsorbable composition to one or more surfaces of a bone fragment and/or to the surface of a bone void to be filled by the fragment, and pressing the fragment into the void.

In some embodiments, the method for stabilizing, repairing, or reapproximating of bone fragments of the present disclosure, comprises intraoperatively mixing the two or more individual reactive putties to form a nonabsorbable composition, optionally dividing the nonabsorbable composition into two or more additional portions, applying a first portion of the nonabsorbable composition to one or more surface(s) of a bone fragment and/or to the surface of a bone void to be filled by the fragment, and pressing the fragment into the void. In some embodiments, the method further comprises applying a second portion of the nonabsorbable composition across the reapproximated surface in the form of a plate or tape. In some embodiments, the method further comprises pressing additional portions of the nonabsorbable composition into each of two or more drill holes located adjacent to each other, or opposite each other across the fracture line, thereby substantially filling each drill hole.

In some embodiments, the method for stabilizing, repairing, or reapproximating of bone fragments of the present disclosure, further comprises stabilizing a surgical screw for stabilizing, repairing, or reapproximating the bone fracture, the method comprising intraoperatively mixing two or more individual reactive putties to form a nonabsorbable composition, filling a drilled or tapped hole with a portion of nonabsorbable composition, and inserting the screw into the nonabsorbable composition before it hardens, or, optionally, setting the screw into the nonabsorbable composition after it hardens.

In some embodiments, the disclosure provides a method for repair of cartilaginous tissue, the method comprising intraoperatively mixing two or more individual reactive putties of the present disclosure, to form a moldable, settable, nonabsorbable polyurethane, polyureaurethane, polyetherurethane or polyetherureaurethane (nonabsorbable composition), and applying the nonabsorbable composition into a void or space formerly occupied by cartilaginous tissue in an amount sufficient to substantially fill the void or space.

In some embodiments, the method for stabilizing, repairing, or reapproximating of bone fragments of the present disclosure, further comprises stabilizing a surgical hardware for stabilizing, repairing, or reapproximating the bone fracture, the method comprising intraoperatively mixing two or more individual reactive putties to form a nonabsorbable composition, optionally dividing the nonabsorbable composition into two or more additional portions, and applying a portion of the nonabsorbable composition between the surface of a bone and the surgical hardware, and/or by applying a portion of the nonabsorbable composition across the surface of the surgical hardware after it has been affixed to the bone. In some embodiments, the method comprises intraoperatively mixing two or more individual reactive putties to form a nonabsorbable composition, and combining the nonabsorbable composition with the surgical hardware, and/or by applying a portion of the nonabsorbable composition across the surface of the surgical hardware to form a nonabsorbable composition/hardware construct, and affixing the nonabsorbable composition/hardware construct is affixed to the bone. In some embodiments, the surgical hardware is a plate, a screw, a mesh, a nail, a cap, a wire, a flap, or a similar surgical device known in the art, or any combination thereof. In some embodiments, the screw is a fenestrated screw that comprises a canula or cavity along the shaft or body of the screw. In some embodiments, the method comprises applying a portion of nonabsorbable composition through the canula or cavity of the screw.

A method of stabilizing a surgical hardware for stabilizing, repairing, or reapproximating a bone fracture, the method comprising the steps of: a) intraoperatively mixing or kneading together a set of at least two reactive putties, A and at least B, of the composition disclosed herein, to form a moldable, settable, non-absorbable polyurethane, polyureaurethane, polyetherurethane or polyetherureaurethane composition at room or body temperature, b) combining the composition with the surgical hardware, and/or applying the mixed or kneaded composition to the surfaces of the bone fracture or the cut surfaces of the sternotomy, and manually reducing the bone fragments while allowing the composition to set; and c) allowing the composition to harden into its fully cured solid form.

In some embodiments of the method of stabilizing a surgical hardware for stabilizing, repairing, or reapproximating a bone fracture disclosed herein, the surgical hardware is any one of a plate, a screw, a mesh, a nail, a cap, a wire, a flap or a combination thereof. In some embodiments of method of stabilizing a surgical hardware for stabilizing, repairing, or reapproximating a bone fracture disclosed herein, the bone fracture is any one of a cranial bone fracture or defect, a pelvic bone fracture or defect or long bone fracture. In some embodiments of the method of stabilizing a surgical hardware for stabilizing, repairing, or reapproximating a bone fracture disclosed herein, the method further comprises applying the mixed or kneaded composition to the surfaces of the bone fracture or the cut surfaces of the sternotomy through a surgical mesh to create a cap or a flap, and manually reducing or reapproximating the bone fragments while allowing the composition to set; and allowing the composition to harden into its fully cured solid form. In some embodiments of the method of stabilizing a surgical hardware for stabilizing, repairing, or reapproximating a bone fracture disclosed herein, the method further comprises customizing the plate or flap by cutting or otherwise machining the mesh prior to or after the non-absorbable composition is applied to the mesh or flap.

In some embodiments, the nonabsorbable compositions of the present disclosure has mechanical properties suitable for drilling and/or accepting a surgical screw without shattering or splintering. In some embodiments, the nonabsorbable compositions provided herein can be used in combination with a surgical hardware, e.g., a screw, a plate, a wire, a rod, or a nail, wherein the composition is used as an adhesive to keep the surgical hardware in contact with a bone. In one embodiment, the nonabsorbable compositions provided herein can be used in combination with a surgical hardware, e.g. a screw, a plate, a wire, a rod, or a nail, wherein the composition is used as a material to drill, affix, or insert the surgical hardware. In some embodiments, the surgical hardware can be a cannulated surgical hardware, wherein the cannulation of the surgical hardware comprises the nonabsorbable compositions disclosed herein. In some embodiments, the cannulated surgical hardware can comprise at least one fenestration to allow the nonabsorbable compositions to extrude from the cannulation of the surgical hardware into the surrounding bone.

Provided herein, is a cannulated bone screw having a shaft, a tip, and a head, with at least a portion of said shaft having threads thereon configured to be inserted into a bone, wherein the screw comprises a cannulation along the shaft comprising an amount of the nonabsorbable compositions disclosed herein within the cannulation, and at least one fenestration disposed along the shaft of the screw and connected to the cannulation of the screw, wherein the fenestration allows the composition to pass through the at least one fenestration of the screw into a bone.

In some embodiments, the cannulated bone screw can be configured for insertion into a hole drilled in a bone. In some embodiments, the cannulated bone screw can be configured for securing in the hole drilled in a bone by an amount of the composition disclosed herein that is in contact with the outer surface of the screw and the walls of the hole. In some embodiments, the cannulated bone screw can be configured for securing into a bone by a surgical plate. In some embodiments, the cannulated bone screw can be configured for securing into a bone by at least one surgical nail. In some embodiments, the cannulated bone screw can be configured for securing to the bone by a nut or a washer.

In some embodiments, the cannulated bone screw can be for use in fracture line reduction between two or more pieces of a bone. In some embodiments, the site of the bone can be a surgical site or a site of injury.

In some embodiments, the bone screw can be for use in a method of stabilizing, repairing, or reapproximating a bone fracture or a sternotomy, disclosed herein. In some embodiments, the method of stabilizing, repairing or reapproximating a bone fracture can be a method of bone surgery of the bone of a jaw, hip, pelvis, knee, ankle, and foot. In some embodiments, the method of bone surgery can be a surgery of phalanx, metacarpals, radius, ulna, fibula, femur, clavicle, humerus, tibia, scapula, vertebra, pelvis, or rib. In some embodiments, the method of bone surgery can be arthroscopy, joint replacement, revision joint surgery, bone fracture repair, debridement, fusion of bones, spine fusion, or osteotomy. In some embodiments, the method of bone surgery can be ankle fracture repair, knee arthroscopy, knee replacement, repair of femoral neck fracture and trochanteric fracture, hip replacement, wrist bone (distal radius) fracture repair, shoulder arthroscopy, laminectomy, lumbar spinal fusion, lower back intervertebral disc surgery, forearm (radius and/or ulna) bone fracture repair, thigh bone (femoral shaft) fracture repair, or an orthodontic surgery.

In some embodiments, the cannulated bone screw can be used for any of the following: joining two pieces of a fractured bone, securing a surgical hardware (e.g., a plate, a rod or a cap or a replacement thereof to a bone), and/or maintaining or positioning one or more bones in a desired anatomical position or orientation.

In some embodiments, the cannulated bone screw can be a cortical screw, a cancellous screw or a Herbert screw. In some embodiments, the cannulated bone screw can be a dynamic hip screw, Acutrak screw, malleolar screw, a locking bolt screw, an interference screw, or a pedicle. In some embodiments, the cannulated bone screw can further comprise a central opening in the outer end of the head of the screw configured for receiving a driving tool for driving the screw into the bone, and for receiving the homogenous composition. In some embodiments, the head of the cannulated bone screw can be threaded, hexagonal, or crossed. In some embodiments, the head of the cannulated bone screw can be threaded. In some embodiments, the head of the cannulated bone screw can be hexagonal. In some embodiments, the head of the cannulated bone screw can be crossed.

In some embodiments, the head of the cannulated bone screw can have a recess of about 2.5 mm to about 4 mm. In some embodiments, the head of the cannulated bone screw can have a recess of 2.5 mm to 4 mm. In some embodiments, the head of the cannulated bone screw can have a recess of 2.5 mm to 3 mm. In some embodiments, the head of the cannulated bone screw can have a recess of 3 mm to 3.5 mm. In some embodiments, the head of the cannulated bone screw can have a recess of 3.5 mm to 4 mm. In some embodiments, the head of the cannulated bone screw can have a recess of 2.5 mm. In some embodiments, the head of the cannulated bone screw can have a recess of 2.8 mm. In some embodiments, the head of the cannulated bone screw can have a recess of 3 mm. In some embodiments, the head of the cannulated bone screw can have a recess of 3.25 mm. In some embodiments, the head of the cannulated bone screw can have a recess of 3.5 mm. In some embodiments, the head of the cannulated bone screw can have a recess of 3.75 mm. In some embodiments, the head of the cannulated bone screw can have a recess of 4 mm.

In some embodiments, the tip of the cannulated bone screw disclosed herein can be non-self-tapping, self-tapping, self-drilling, or self-tapping and self-drilling. A “self-tapping” and “a self-drilling” bone screw as described herein means that the tip of the screw will make or drill a hole and will cut the channel for the thread.

In some embodiments, the cannulated bone screw can have a thread pitch between 9.0 TPI (thread per inch) to 40 TPI. In some embodiments, the cannulated bone screw can have a thread pitch between 9.0 TPI to 20 TPI. In some embodiments, the cannulated bone screw can have a thread pitch between 20 TPI to 30 TPI. In some embodiments, the cannulated bone screw can have a thread pitch between 30 TPI to 40 TPI. The term “thread pitch” as used herein means the number of threads present per inch of the screw.

In some embodiments, the cannulated bone screw can have a length of about 8 mm to about 150 mm. In some embodiments, the cannulated bone screw can have a length of 8 mm to 20 mm. In some embodiments, the cannulated bone screw can have a length of 20 mm to 30 mm. In some embodiments, the cannulated bone screw can have a length of 30 mm to 40 mm. In some embodiments, the cannulated bone screw can have a length of 40 mm to 50 mm. In some embodiments, the cannulated bone screw can have a length of 50 mm to 75 mm. In some embodiments, the cannulated bone screw can have a length of 75 mm to 80 mm. In some embodiments, the cannulated bone screw can have a length of 80 mm to 100 mm. In some embodiments, the cannulated bone screw can have a length of 100 mm to 125 mm. In some embodiments, the cannulated bone screw can have a length of 125 mm to 150 mm.

In some embodiments, the cannulated bone screw can have an outer diameter of about 3.5 mm to about 7.5 mm. In some embodiments, the cannulated bone screw can have an outer diameter of 3.5 mm to 7.5 mm (i.e., 3.5 to 4 mm, 3.5 to 3.75 mm, 3.75 to 4 mm) and a shaft diameter of 2.0 mm to 5.0 mm (i.e., 2.0 to 2.5 mm, 2.5 to 3.0 mm, 3.0 to 3.5 mm, or 3.5 to 4.0 mm).

In some embodiments, the cannulated bone screw can be made of titanium, stainless steel, or a bioabsorbable material. In some embodiments, the bioabsorbable material is polylactic acid, polyglycolic acid, poly-L-lactic acid, or polylactic acid and tricalcium phosphate.

In some embodiments, the cannulated bone screw can be inserted into a bone of a hip, pelvis, knee, ankle, and/or foot. In some embodiments, the cannulated bone screw can be inserted into a bone or portion of phalanx, metacarpals, radius, ulna, fibula, femur, clavicle, humerus, tibia, scapula, vertebra, pelvis, or rib.

Provided herein is a method of delivering a nonabsorbable composition of the present disclosure into a site in a bone of a patient in need thereof, the method comprising: a) providing the cannulated bone screw disclosed herein; b) inserting the cannulated bone screw into the bone of the patient; and c) allowing the amount of the nonabsorbable composition within the bone screw to be delivered into the bone.

In some embodiments, the step of providing the cannulated bone screw disclosed herein further comprises delivering an amount of the nonabsorbable composition disclosed herein, into the cannulation of the bone screw. In some embodiments, the delivering of an amount of the nonabsorbable composition disclosed herein into the cannulation can be done by means of a syringe, wherein the syringe is connected to the central opening in the outer end of the head of the screw. In some embodiments, the delivering of an amount of the nonabsorbable composition disclosed herein into the cannulation of the cannulated bone screw can be done from a reservoir that is connected to the central opening in the outer end of the head of the screw by means of a pump.

In some embodiments, the inserting of the cannulated bone screw into the bone of the patient can be done by drilling the screw into the bone. In some embodiments, inserting the cannulated bone screw into the bone of the patient can be done by: a) drilling a hole in the bone of the patient; b) filling the hole with a nonabsorbable composition disclosed herein; c) allowing the composition in the drilled hole of the bone to solidify; and d) inserting the screw disclosed herein through the solidified nonabsorbable composition in the drilled hole of the bone.

In some embodiments, the inserting of the cannulated bone screw into the bone of the patient can be done by means of an orthopedic screwdriver, an orthopedic electric drilling device, an orthopedic mechanical drilling device, an orthopedic tap handle, a bone mallet, or equivalent thereof.

In some embodiments, the inserting of the cannulated bone screw into the bone of the patient can be done by placing a surgical plate between the screw and the bone. In some embodiments, the inserting of the cannulated bone screw into the bone of the patient can be done by placing a surgical washer or a nut between the screw and the bone. In some embodiments, the inserting of the cannulated bone screw into the bone of the patient can be done by using a surgical guide wire with the bone screw.

In some embodiments, the inserting of the cannulated bone screw into the bone of the patient further comprises securing the screw in the bone by means of at least one surgical nail.

In some embodiments, the method of delivering a bone hemostatic and adhesive nonabsorbable composition of the present disclosure into a site in a bone disclosed herein, further comprises delivering additional amounts of the nonabsorbable composition disclosed herein into the cannulation of the bone screw after inserting the bone screw into the bone. In some embodiments, the method of delivering a bone hemostatic and adhesive nonabsorbable composition of the present disclosure, into a site in a bone disclosed herein, can be carried out in combination with a method of bone surgery. In some embodiments, the method of bone surgery can be a surgery of the bone of a jaw, hip, pelvis, knee, ankle, and foot. In some embodiments, the method of bone surgery can be a surgery of phalanx, metacarpals, radius, ulna, fibula, femur, clavicle, humerus, tibia, scapula, vertebra, pelvis, or rib. In some embodiments, the method of bone surgery can be arthroscopy, joint replacement, revision joint surgery, bone fracture repair, debridement, fusion of bones, spine fusion, or osteotomy. In some embodiments, the method of bone surgery can be ankle fracture repair, knee arthroscopy, knee replacement, repair of femoral neck fracture and trochanteric fracture, hip replacement, wrist bone (distal radius and/or ulna) fracture repair, shoulder arthroscopy, laminectomy, lumbar spinal fusion, lower back intervertebral disc surgery, forearm (radius and/or ulna) bone fracture repair, thigh bone (femoral shaft) fracture repair, or an orthodontic surgery.

In some embodiments, the composition of the present disclosure can be for use in a method of securing surgical hardware into a bone of a subject in need thereof, wherein the surgical hardware has a body and an opening at a point of the body, wherein the surgical hardware is cannulated inside the body of the hardware, and comprises at least one fenestration along the body of the hardware for receiving the composition, wherein the method comprises: a) inserting the surgical hardware into a site on the bone; b) delivering the composition hardware inside through the opening into the cannulation in the body of the surgical hardware; c) allowing the composition to pass through the fenestration of the surgical hardware into an area surrounding the hardware in the bone; and d) allowing the composition to harden to secure the surgical hardware to the bone. In some embodiments, the surgical hardware can be a screw, a plate, a wire, a rod, a nail, or equivalent thereof.

In some embodiments, the composition of the present disclosure can be for use in a method for stabilizing, repairing, or reapproximating of bone fragments of the present disclosure, wherein the method comprises: a) intraoperatively mixing or kneading together a set of two or more reactive putties, A and at least B, of any of the compositions of the present disclosure, to form a moldable, settable, nonabsorbable polyurethane, polyureaurethane, polyetherurethane or polyetherureaurethane composition (nonabsorbable composition) at room or body temperature; b) applying the mixed or kneaded composition to the surfaces of the bone fracture or the cut surfaces of the sternotomy through a surgical mesh to create a cap or a flap, and manually reducing or reapproximating the bone fragments while allowing the composition to set; and c) allowing the composition to harden into its fully cured solid form. In some embodiments, the method further comprises customizing the plate or flap by cutting or otherwise machining the mesh prior to or after the nonabsorbable composition is applied to the mesh or flap.

The present disclosure also provides a plurality of biocompatible, settable putties which, upon mixing, react to form a cured final composition at room or body temperature over a period time, the final composition being nonabsorbable under physiological conditions, wherein the plurality of compositions comprise at least two putties of the compositions disclosed herein. In some embodiments, each of the at least two putties of the plurality of biocompatible, settable putties of the present disclosure, is physically separated from the other components of the set within the package, and optionally, from other sets of components. In some embodiments, the plurality of biocompatible, settable putties of the present disclosure, are sterile. In some embodiments, the plurality of biocompatible, settable putties is adapted to permit the removal of one set of components at a time while leaving the remaining sets in a sealed, sterile, environment. In some embodiments, the plurality of biocompatible, settable putties comprises at least two putties of the composition disclosed herein, are in the form of surgical kits and packages. In one embodiment, each component is physically separated from the other components of its set within the package by means of a compartment or plurality of compartments in the package. In some embodiments, each of the at least two putties of the plurality of biocompatible, settable putties may optionally be separated from other putties by perforations allowing the set to be conveniently separated either before or after opening and removing the contents.

In some embodiments, the plurality of biocompatible, settable putties comprises depressions or wells in a heat-sealable metal foil-based sheet. In some embodiments, the plurality of biocompatible, settable putties is flexible and separated by at least one breakable seal adapted to allow the at least two putties of the plurality of the putties to be mixed together when the seal is broken. In some embodiments, the at least two putties of the plurality of biocompatible, settable putties are in the form of one or more syringes, preferably one or more foil-enclosed syringes. In one embodiment, the at least two putties of the plurality of biocompatible, settable putties are in the form of a single syringe, preferably a foil-enclosed syringe, adapted to maintain individual component putties of a set separated from each other within the single syringe. In one embodiment, the at least two putties of the plurality of biocompatible, settable putties are in the form of a plurality of syringes and each syringe contains a single putty of the plurality of biocompatible, settable putties. In some embodiments, each of the at least two putties of the plurality of biocompatible, settable putties comprises one or more surfaces in contact with another putty, the one or more surface(s) comprising or consisting of a low surface energy material selected, for example, from the group consisting of polytetrafluoroethylene (PTFE), silicone, polypropylene, polyethylene, and polystyrene.

In some embodiments, the plurality of biocompatible, settable putties further comprise an outer, heat-sealable, preferably water impermeable, or water resistant envelope completely surrounding the plurality of biocompatible, settable putties, and a desiccant. In some embodiments, the outer envelope is a heat-sealed, water impermeable or water resistant foil package.

The present disclosure also provides a settable, nonabsorbable putty composition (nonabsorbable composition) formed by mixing the at least two putties of the present disclosure. In some embodiments, the nonabsorbable composition of the present disclosure comprises 5% to 35% of a polyisocyanate component, 2.5% to 42.5% of a polyol/polyamine component, 30% to 90% of one or more particulate matter(s), and 0% to 6.5% of one or more additive material(s), based upon the weight of the composition.

In some embodiments, the nonabsorbable composition of the present disclosure comprises 5% to 35% (e.g., 5% to 10%, 10% to 15%, 15% to 20%, 20% to 25%, 25% to 30% or 30% to 35%) of a polyisocyanate component, based upon the weight of the composition. In some embodiments, the nonabsorbable composition of the present disclosure comprises 2.5% to 42.5% (e.g., 2.5% to 5%, 5% to 7.5%, 7.5% to 10%, 10% to 12.5%, 12.5% to 15%, 15% to 17.5%, 17.5% to 20%, 20% to 22.5%, 22.5% to 25%, 25% to 27.5%, 27.5% to 30%, 30% to 32.5%, 32.5% to 35%, 35% to 37.5%, 37.5% to 40%, or 40% to 42.5%) of a polyol/polyamine component, based upon the weight of the composition. In some embodiments, the nonabsorbable composition of the present disclosure comprises 30% to 90% (e.g., 30% to 35%, 35% to 40%, 40% to 45%, 45% to 50%, 50% to 55%, 55% to 60%, 60% to 65%, 65% to 70%, 70% to 75%, 75% to 80%, 80% to 85%, or 85% to 90%) of one or more particulate matter(s), based upon the weight of the composition. In some embodiments, the nonabsorbable composition of the present disclosure comprises 0% to 6.5% (e.g., 0% to 2%, 2% to 4% or 4% to 6.5%) of one or more additive material(s), based upon the weight of the composition.

In some embodiments, the nonabsorbable composition of the present disclosure comprises 12.5% to 30% of a polyisocyanate component, 15% to 35% of a polyol/polyamine component, and 35% to 72.5% of one or more particulate matter(s), based upon the weight of the composition. In some embodiments, the nonabsorbable composition of the present disclosure comprises 12.5% to 30% of a polyisocyanate component (e.g., 12.5% to 15%, 15% to 17.5%, 17.5% to 20%, 20% to 22.5%, 22.5% to 25%, 25% to 27.5%, or 27.5% to 30%), based upon the weight of the composition. In some embodiments, the nonabsorbable composition of the present disclosure comprises 15% to 35% of a polyol/polyamine component (e.g., 15% to 17.5%, 17.5% to 20%, 20% to 22.5%, 22.5% to 25%, 25% to 27.5%, 27.5% to 30%, 30% to 32.5%, or 32.5% to 35%), based upon the weight of the composition. In some embodiments, the nonabsorbable composition of the present disclosure comprises 35% to 72.5% of one or more particulate matter(s) (e.g., 35% to 37.5%, 37.5% to 40%, 40% to 42.5%, 42.5% to 45%, 45% to 47.5%, 47.5% to 50%, 50% to 52.5%, 52.5% to 55%, 55% to 57.5%, 57.5% to 60%, 60% to 62.5%, 62.5% to 65%, 65% to 67.5%, 67.5% to 70%, or 70% to 72.5%), based upon the weight of the composition.

In some embodiments, the nonabsorbable composition of the present disclosure comprises 8.5% to 22.5% of a polyisocyanate component, 8.5% to 14% of a polyol/polyamine component, and 65% to 85% of one or more particulate matter(s), based upon the weight of the composition. In some embodiments, the nonabsorbable composition of the present disclosure comprises 8.5% to 22.5% (e.g., 8.5% to 11%, 11% to 13.5%, 13.5% to 16%, 16% to 18.5%, 18.5% to 21%) of a polyisocyanate component, based upon the weight of the composition. In some embodiments, the nonabsorbable composition of the present disclosure comprises 8.5% to 14% (e.g., 8.5% to 10%, 12% to 13%, or 13% to 14%) of a polyol/polyamine component, based upon the weight of the composition. In some embodiments, the nonabsorbable composition of the present disclosure comprises 65% to 85% (e.g., 65% to 70%, 70% to 75%, 75% to 80%, or 80% to 85%) of one or more particulate matter(s), based upon the weight of the composition.

In some embodiments, the nonabsorbable composition of the present disclosure comprises 13.5% to 25% of a polyisocyanate component, 4% to 20% of a polyol/polyamine component, and 55% to 78.5% of one or more particulate matter(s), based upon the weight of the composition.

In some embodiments, the nonabsorbable composition of the present disclosure comprises 13.5% to 25% (e.g., 13.5% to 15%, 15% to 17.5%, 17.5% to 20%, 20% to 22.5%, or 22.55 to 25%) of a polyisocyanate component, based upon the weight of the composition. In some embodiments, the nonabsorbable composition of the present disclosure comprises 4% to 20% (e.g., 4% to 8%, 8% to 12%, 12% to 16%, or 16% to 20%) of a polyol/polyamine component, based upon the weight of the composition. In some embodiments, the nonabsorbable composition of the present disclosure comprises 55% to 78.5% (e.g. 55% to 57.5%, 57.5% to 60%, 60% to 62.5%, 62.5% to 65%, 65% to 67.5%, 67.5% to 70%, 70% to 72.5%, 72.5% to 75%, or 75% to 78.5%) of one or more particulate matter(s), based upon the weight of the composition.

### EXAMPLES

### Examples of Nondegradable HPC Compositions and Reactive Putties

The following provides non-limiting examples of exemplary nondegradable settable and hemostatic HPC compositions and their reactive components (putties/pastes), and methods of making the same. The following also describe their physical and chemical properties of the exemplified compositions provided herein, making them suitable for use in the surgical methods described herein.

The settable reactive components (putties/pastes) of the nonabsorbable composition described herein, were produced by mixing a liquid polyisocyanate component and a polyol component with filler particles. Enough solid filler particles/granules were added to establish suitable handling properties. For all isocyanate putties, 1,1,1-tris-(4-isocyanatophenoxymethyl)-propane (TMPI), was the polyisocyanate used. All polyol-based putties used a polyol solution that consisted of trimethylolpropane ethoxylate (TMPE), tetrakis(2-hydroxypropyl)ethylenediamine (TKP), triethanolamine (TEA), and/or butanediol (BDO). In certain embodiments, combinations of the aforementioned polyols were used. Additionally, in certain compositions of the present invention, the polyisocyanate and a polyol were partially pre-reacted with each other to enhance physical handling characteristics of the material.

In some example compositions described herein, three different filler types were used to create various formulations of the present invention, including calcium phosphate particles (CaP), stainless steel particles (SS), and demineralized bone matrix particles (DBM). The amounts of these particulate fillers generally varied between 30-80 wt % of the final composite material composition. Also, the size of these particles was varied to evaluate the effect filler particle size has on the properties of the present invention.

For formulations comprising CaP particles, varying phases of CaP were used (e.g., hydroxyapatite and/or β-tricalcium phosphate). Additionally, there were varying particle sizes used, ranging from nanoparticles to macroscopic granules.

For formulations comprising hydroxyapatite (HA) nano particles, enough HA was weighed in a mixing container so there would be about 40 wt % HA in the final mixture. The isocyanate was then added to the mixing container, where the mixture was stirred to homogeneity. A combination of TEA and TKP was then added to the mixing container and the mixture was stirred to homogeneity, resulting in a thick paste. The mixture was then subsequently applied to a Sawbone surface. The mixture was spreadable for ˜2 minutes, after which it hardened into a stiff composite material that was no longer spreadable.

In a similar exemplified composition, milled HA powder was weighed in a mixing container so there would be about 50 wt % HA powder in the final mixture. The polyisocyanate component was added and the mixture was subsequently stirred to homogeneity. Following this mixing, a mixture of TEA and TKP was added to the mixing container and mixed to homogeneity, resulting in a less viscous paste than the paste resulting from the use of HA nanoparticles. While the hardening time and overall spreadability was similar between the two pastes, the use of larger milled HA particles caused an overall decrease in viscosity relative to the use of nanoparticles.

In another exemplified composition, a fraction of the polyisocyanate was reacted with a polyol to enhance physical handling prior to reacting the entire isocyanate present. Milled HA was weighed in a mixing container so there would be about 40 wt % HA in the final mixture. A combination of TMPI and TMPE (so that ˜10% of the TMPI was reacted) was then added to the mixing container, and the mixture was stirred to homogeneity. The container was then sealed and allowed to sit for several hours. The resulting mixture formed a soft, sticky putty that could be manipulated by hand. In a similar exemplified formulation, HA was weighed in a mixing container so there would be about 60 wt % HA in the final mixture. A combination of TMPE and TMPI (so that ˜15% of the TMPE was be reacted) was then added to the mixing container and the mixture was stirred to homogeneity. The mixing container was then sealed and allowed to sit for several hours. The resulting mixture was stiff and slightly crumbly, but could be manipulated by hand. These results disclosed herein show that having excess, unreacted TMPI resulted in a stress-relaxing composite mixture that can be manipulated by hand, whereas having excess TMPE results in a mixture that retains its shape indefinitely and can be manipulated by hand.

In a similar exemplified composition, DBM was weighed in a mixing container so there would be about 50 wt % DBM in the final mixture. A combination of TMPI and TMPE (so that ˜10% of the TMPI was be reacted) was added to the mixing container and the mixture was stirred to homogeneity. The mixing container was then sealed and allowed to sit for several hours. The resulting material was a granular putty material. The material was hand-moldable, but less cohesive than the material previously described, likely due to the larger size and lower density of the particle filler used.

Using the same technique as used for making the exemplified compositions described above, approximately 90% of the estimated total mass of stainless-steel particles were weighed in a mixing container. A combination of TMPI and TMPE (so that ˜10% of the TMPI was be reacted) was added to the mixing container and the mixture was stirred to homogeneity. The mixing container was then sealed and allowed to sit for several hours. The resulting material was a soft putty material. The putty was more cohesive and softer than the putty made using DBM as the filler material. Additionally, the material was uniformly gray and noticeably heavier than the putties previously made.

By virtue of the ether linkages in TMPI, the polyurethane composite materials containing calcium phosphate or stainless-steel particles are not susceptible to hydrolytic degradation. To this end, an experiment was performed to evaluate any hydrolytic degradation of the present invention that could occur. Composite materials, similar to what has been described previously, were prepared by combining milled HA powder, TMPI, and TMPE in a mixing container and stirring to homogeneity. The resulting mixture was formed into uniform shapes that were subsequently submerged in a phosphate buffered saline solution under accelerated aging conditions (e.g., at 70° C.). The mass loss of the material was evaluated and converted to a real-time equivalence using the Arrhenius equation. At two years of real-time equivalence aging, the composite material showed no signs of hydrolytic degradation. There was no significant mass loss and no statistically significant decrease in mechanical properties for the duration of the accelerated aging study. These results were compared to results of a similar aging study performed on an absorbable polyurethane-based bone hemostat. The absorbable material showed significant mass loss and mechanical property deterioration over the course of the 2-year real-time equivalent study.

### General Putty Observations

Table 1 provides a summary of the putty compositions disclosed herein. The table describes a Putty A and Putty B for each formulation. The primary liquid component of Putty A is TMPI and the primary liquid component for Putty B is a polyol. In some cases, the primary liquid component of the putties was partially reacted with their counterpart (i.e., TMPI was partially reacted with the polyol or the polyol was partially reacted with TMPI) as a means to adjust the viscoelasticity (i.e., handling properties) of the putties.

TABLE 1

| Trial | Putty | Liquid Reactive | Additive | Component Putty | Putty C (Mixed Putty) |
|---|---|---|---|---|---|
| Composition | (A or B) | Components (wt %) | Filler (wt %) | Observations | Observations |
| 1 | A | TMPI (60%) | HA, 10 μm | low viscosity paste | Pastes were stirred to |
| | | | (40%) | | homogeneity with spatula. |
| | B | Polyol (70%) | HA, 10 μm | low viscosity paste | The mixed product remained |
| | | | (30%) | | a viscous paste for 3-4 |
| | | | | | minutes before beginning to |
| | | | | | harden. The mixed product |
| | | | | | seemed fully hardened after |
| | | | | | 24 hours. |
| 2 | A | TMPI (60%) | HA np (40%) | Thick, shape- | Thicker pastes likely a result |
| | | | | retaining paste | of using nanoparticles, rather |
| | B | Polyol (70%) | HA np (30%) | Thick, shape- | than the microparticles used |
| | | | | retaining paste | in example 1. Pastes were |
| | | | | | stirred to homogeneity with |
| | | | | | spatula. The mixing took |
| | | | | | longer than in example 1, but |
| | | | | | hardening began at about the |
| | | | | | same time as previous |
| | | | | | example. Mixed product |
| | | | | | seemed fully hardened at 24 |
| | | | | | hours. |
| 3 | A | TMPI (25%) | HA, 10 μm | Soft, sticky putty. | Putties were able to be mixed |
| | | | (75%) | Putty also fully | by hand, but stuck to gloves |
| | | | | hardened within a | in the process. Product was |
| | | | | few days of storing | workable for about 10 |
| | | | | it in sealed | minutes, but appeared fully |
| | | | | container. Likely a | hardened after 24 hours. |
| | | | | result of a reaction | |
| | | | | between TMPI and | |
| | | | | HA particles. | |
| | B | Polyol (30%) | HA, 10 μm | Soft putty, but not | |
| | | | (70%) | sticky. | |
| 4 | A | TMPI (30%) | β-TCP, 10 | Same overall | Putties were able to be mixed |
| | | | μm (70%) | consistency as | by hand, but stuck to gloves |
| | | | | example 3, but | in the process. Product was |
| | | | | remained stable for | workable for about 10 |
| | | | | longer. | minutes, but appeared fully |
| | B | Polyol (30%) | HA, 10 μm | Soft putty, but not | hardened after 24 hours. |
| | | | (70%) | sticky. | |
| 5 | A | TMPI (36%), Polyol | HA, 10 μm | Good handling | Component putty B was a |
| | | (2%) | (62%) | properties | little crumbly, but overall, the |
| | B | Polyol (16%), TMPI | HA, 10 μm | Good handling | putties mixed well together. |
| | | (3%) | (81%) | properties, slightly | Product was workable for |
| | | | | crumbly | about 5 minutes, and |
| | | | | | appeared fully hardened after |
| | | | | | 24 hours. |
| 6 | A | TMPI (16%), Polyol | HA/β-TCP, | Sticky, but hand | Stuck to gloves during |
| | | (2%) | 250-500 μm | moldable | mixing. Mixed product |
| | | | (82%) | | remained soft for about 10 |
| | B | Polyol (23%), TMPI | HA/β-TCP, | Soft putty, but hand | minutes and appeared to be |
| | | (3%) | 250-500 μm | moldable | fully hardened after 24 hours. |
| | | | (74%) | | |
| 7 | A | TMPI (31%), Polyol | HA/β-TCP, | Good handling | Putties were easily hand- |
| | | (4%) | 250-500 μm | properties | mixed and subsequently |
| | | | (41%) | | workable for about 5 minutes. |
| | | | β-TCP, 10 | | Appeared totally hardened |
| | | | μm (24%) | | after 24 hours. |
| | B | Polyol (18%), TMPI | HA, 10 μm | Good handling | |
| | | (5%) | (77%) | properties | |
| 8 | A | TMPI (29%), Polyol | SS, 10 μm | Soft putty, good | Putties mixed well and the |
| | | (3%) | (68%) | handling properties | mixed product was workable |
| | B | Polyol (7%), TMPI | SS, 10 μm | Good handling | for up to 5 minutes. Appeared |
| | | (2%) | (91%) | properties | totally hardened after 24 |
| | | | | | hours. |
| 9 | A | TMPI (41%), Polyol | DBM (54%) | Granular, good | Slightly crumbly when |
| | | (5%) | | handling properties | mixing putties, but was |
| | B | Polyol (31%), TMPI | DBM (64%) | Granular, good | workable for about 10 |
| | | (5%) | | handling properties | minutes. Appeared hardened |
| | | | | | after 24 hours. |

TMPI is the isocyanate used in every formulation listed here
Polyol is any of the aforementioned polyols (of combinations thereof) described herein
HA = hydroxyapatite
β-TCP = β-tricalcium phosphate
HA/β-TCP = biphasic hydroxyapatite and β-tricalcium phosphate granules
SS = stainless steel
DBM = demineralized bone matrix

### Mixed Putty Observations

Polyurethane/calcium phosphate, polyurethane/stainless steel, polyurethane/demineralized bone matrix composite materials were fabricated by combining a polyisocyanate and a polyol (or in some cases a pre-reacted forms of these components) putties in approximately a 1:1 molar ratio of polyisocyanate:polyol. After mixing the two reactive putties, generally the combined putty was mildly exothermic, becomes slightly softer and stickier, and eventually hardened over time. All individual putties discussed herein could be manipulated by hand and were workable over cut bone surfaces. Similarly, the combined putties discussed herein are spreadable for a short period of time before undergoing hardening and becoming a rigid material.

The examples provided herein clearly define the physical and chemical characteristics of the various nonabsorbable constituent putty compositions (putties A and B) provided herein, and the resulting settable nonabsorbable compositions (mix putty or putty C) formed by mixing the constituent putty compositions. The observations of the resulting settable, nonabsorbable compositions show that the nonabsorbable compositions of the present disclosure have the characteristics suitable for use in the methods described herein.

### EQUIVALENTS

Those skilled in the art will recognize or be able to ascertain using no more than routine experimentation, many equivalents to the specific embodiments of the invention described herein. Such equivalents are intended to be encompassed by the following claims.

All references cited herein are incorporated herein by reference in their entirety and for all purposes to the same extent as if each individual publication or patent or patent application was specifically and individually indicated to be incorporated by reference in its entirety for all purposes.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

Unless indicated otherwise, all percentages by are percentages by weight, parts are parts by weight, temperature is in degrees Celsius, and pressure is at or near atmospheric. There are numerous variations and combinations of reaction conditions (e.g., component concentrations, desired solvents, solvent mixtures, temperatures, pressures, and other reaction ranges and conditions) that can be used to optimize the product purity and yield obtained from the described process. Only reasonable and routine experimentation will be required to optimize such process conditions.

What is claimed is:

1. A settable nonabsorbable composition comprising a set of at least two reactive putties, A and at least B, which, upon mixing together, react and cure into a final hardened form selected from the group consisting of polyurethane, polyureaurethane, polyetherurethane and polyetherureaurethane, over a period of time at room or body temperature, each putty being physically separated from the other putty of the composition;
wherein putty A comprises 10-70% of a polyisocyanate component, 0-5% of a polyol or a polyamine component, 30-85% of one or more particulate material(s), and 0-8% of one or more additive material(s), based upon total weight of putty A;
wherein putty B comprises 0-5% of a polyisocyanate component, 5-80% of a polyol or a polyamine component, 30-95% of one or more particulate material(s), and 0-5% of one or more additive material(s), based upon the weight of putty B; and
wherein the polyol or polyamine component of putty A or putty B or both comprise one or more nonabsorbable, non-hydrolysable crosslinker(s).

2. The composition of claim 1, wherein
putty A comprises 25-60% of a polyisocyanate component, and 40-75% of one or more particulate materials(s); based upon the weight of putty A; and
putty B comprises 30-70% of a polyol and/or a polyamine component, and 30-70% of one or more particulate material(s), based upon the weight of putty B.

3. The composition of claim 1, wherein
putty A comprises 15-40% of a polyisocyanate component, 2-3% of a polyol and/or a polyamine component, and 60-85% of one or more particulate material(s), based upon the weight of putty A; and
putty B comprises 2-5% of a polyisocyanate compound, 15-25% of a polyol and/or a polyamine component, and 70-85% of one or more particulate material(s), based upon the weight of putty B.

4. The composition of claim 1, wherein
putty A comprises 25-45% of a polyisocyanate component, 3-5% of a polyol and/or a polyamine component, and 50-65% of one or more particulate material(s), based upon the weight of putty A; and
putty B comprises 2-5% of an polyisocyanate compound, 5-35% of a polyol and/or a polyamine component, and 60-92% of one or more particulate material(s), based upon the weight of putty B.

5. The composition of any one of claims 1-4, wherein the nonabsorbable, non-hydrolysable cross-linker is one or more polyol(s) selected from the group consisting of trimethylolpropane ethoxylate, triethanolamine, glycerol, pentaerythritol, a trifunctional castor oil-based polyol, trimethylolpropane polyol, tetrakis(2-hydroxyethyl)ethylenediamine or tetrakis(2-hydroxypropyl)ethylenediamine.

6. The composition of claim 5, wherein the trimethylolpropane ethoxylate is a trimethylolpropane ethoxylate (TMPE) of molecular weight 450, and/or a TMPE of molecular weight 170.

7. The composition of any one of claims 1-6, wherein the polyisocyanate is a non-hydrolysable polyisocyanate.

8. The composition of any one of claims 1-7, wherein the polyisocyanate is a diisocyanate, triisocyanate, or tetraisocyanate.

9. The composition of any one of claims 1-4, wherein the polyamine is selected from ethylene diamine, propane diamine, butane diamine, cyclopentane diamine, cyclohexane diamine, and hexamethylene diamine.

10. The composition of any one of claims 1-9, wherein the particulate material is calcium phosphate, siliconized calcium phosphate, substituted calcium phosphates (substituted with magnesium, strontium, or silicate), calcium pyrophosphate, hydroxyapatite, polymethyl methacrylate, or tricalcium phosphate, or any combination thereof.

11. The composition of any one of claims 1-10, wherein the additive is a colorant, an antioxidant, an active chemical hemostat, a steroid, calcium stearate, tocopheryl acetate, triacetin, a nonabsorbable plasticizer, other therapeutic agent(s) or any combination thereof.

12. The composition of claim 11, wherein the active chemical hemostat is a blood clot-inducing agent selected from a group consisting of prothrombin, thrombin, fibrinogen, fibrin or any combination thereof.

13. The composition of any one of claims 1-12, further comprising one or more antibiotic.

14. The composition of claim 13, wherein the one or more antibiotic is selected from the group consisting of tobramycin, vancomycin, penicillin, methicillin, oxacillin, ampicillin, amoxicillin, a cephalosporin, imipenem, meropenem, bacitracin, neomycin, daptomycin, gentamicin, streptomycin, kanamycin, tetracycline, doxycycline, minocycline, tigecycline, linezolid, chloramphenicol, erythromycin, azithromycin, telithromycin, clindamycin, prisintamycin, metronidazole, ciprofloxacin, norfloxacin, morifloxacin, rifampin, isoniazid, trimethoprim, sulfamethoxazole, and sulfadoxin.

15. A method of stabilizing a bone fracture or reapproximating a sternotomy, the method comprising the steps of:
a) intraoperatively mixing or kneading together a set of at least two reactive putties, A and at least B, of the composition of any one of claims 1-14, to form a moldable, settable, nonabsorbable polyurethane, polyureaurethane, polyetherurethane or polyetherureaurethane composition at room or body temperature,
b) applying the mixed or kneaded composition to the surfaces of the bone fracture or the cut surfaces of the sternotomy, and manually reducing the bone fragments while allowing the composition to set; and
c) allowing the composition to harden into its fully cured solid form.

16. The method of claim 15, wherein step (b) further comprises applying a portion of the composition across the surface of a surgical hardware to create a composition-hardware construct, and affixing the composition-hardware construct to the surfaces of the bone fracture or the cut surfaces of the sternotomy.

17. A method of stabilizing a surgical hardware for stabilizing, repairing, or reapproximating a bone fracture, the method comprising the steps of:
a) intraoperatively mixing or kneading together a set of at least two reactive putties, A and at least B, of the composition of any one of claims 1-14, to form a moldable, settable, nonabsorbable polyurethane, polyureaurethane, polyetherurethane or polyetherureaurethane composition at room or body temperature,
b) combining the composition with the surgical hardware, and/or applying the mixed or kneaded composition to the surfaces of the bone fracture or the cut surfaces of the sternotomy, and manually reducing the bone fragments while allowing the composition to set; and
c) allowing the composition to harden into its fully cured solid form.

18. The method of any one of claims 16-17, wherein the surgical hardware is any one of a plate, a screw, a mesh, a nail, a cap, a wire, a flap or a combination thereof.

19. The method of claim 18, wherein the bone fracture is any one of a cranial bone fracture or defect, a pelvic bone fracture or defect or long bone fracture.

20. A plurality of biocompatible, settable putties which, upon mixing, react to form a cured final composition at room or body temperature over a period time, the final composition being nonabsorbable under physiological conditions, wherein the plurality of putties, comprises the at least two putties of the composition of any one of claims 1-14.

21. A settable nonabsorbable putty composition formed by mixing the at least two putties of the composition of any one of claims 1-14.
